SEQUENCE LISTING (25 sequences)

SEQ ID NO: 1 - RNA, 15 nt, synthetic construct
  source 1..15: mol_type = other RNA
ctgtcttctctggac

SEQ ID NO: 2 - RNA, 23 nt, synthetic construct
  source 1..23: mol_type = other RNA
acattccactgtcttctctggac

SEQ ID NO: 3 - RNA, 23 nt, synthetic construct
  source 1..23: mol_type = other RNA
  modified_base order(1,4,7,11,14,17,20,23): mod_base = OTHER; note = Locked nucleic acids
  modified_base order(2..3,5..6,8..10,12..13,15..16,18..19,21..22): mod_base = OTHER; note = 2-Prime-O-methyl nucleotides
  modified_base 1..4: mod_base = OTHER; note = Phosphorothioate internucleotide linkage
  modified_base 20..23: mod_base = OTHER; note = Phosphorothioate internucleotide linkage
  modified_base order(4,14,17): mod_base = OTHER; note = Thymine
acattccactgtcttctctggac

SEQ ID NO: 4 - RNA, 31 nt, synthetic construct
  source 1..31: mol_type = other RNA
  modified_base order(1,3,5,9,11,13,15,19,22,25,28,31): mod_base = OTHER; note = Locked nucleic acids
  modified_base order(2,4,6..8,10,12,14,16..18,20..21,23..24,26..27,29..30): mod_base = OTHER; note = 2-Prime-O-methyl nucleotides
  modified_base order(1,3,5,11,13,22,25): mod_base = OTHER; note = Thymine
tttttcgacgtcttatctgtcttctctggac

SEQ ID NO: 5 - RNA, 31 nt, synthetic construct
  source 1..31: mol_type = other RNA
  modified_base order(1,3,5,9,11,13,15,19,22,25,28,31): mod_base = OTHER; note = Locked nucleic acids
  modified_base order(2,4,6..8,10,12,14,16..18,20..21,23..24,26..27,29..30): mod_base = OTHER; note = 2-Prime-O-methyl nucleotides
  modified_base order(5,13,22,25): mod_base = OTHER; note = Thymine
ccattagaacactccactgtcttctctggac

SEQ ID NO: 6 - RNA, 31 nt, synthetic construct
  source 1..31: mol_type = other RNA
  modified_base order(1,3,5,9,11,13,15,19,22,25,28,31): mod_base = OTHER; note = Locked nucleic acids
  modified_base order(2,4,6..8,10,12,14,16..18,20..21,23..24,26..27,29..30): mod_base = OTHER; note = 2-Prime-O-methyl nucleotides
  modified_base order(3,13,22,25): mod_base = OTHER; note = Thymine
acttctaaacattccactgtcttctctggac

SEQ ID NO: 7 - RNA, 31 nt, synthetic construct
  source 1..31: mol_type = other RNA
  modified_base order(1,3,5,9,11,13,15,19,22,25,28,31): mod_base = OTHER; note = Locked nucleic acids
  modified_base order(2,4,6..8,10,12,14,16..18,20..21,23..24,26..27,29..30): mod_base = OTHER; note = 2-Prime-O-methyl nucleotides
  modified_base order(9,11,13,22,25): mod_base = OTHER; note = Thymine
gtcgattttgtttacactgtcttctctggac

SEQ ID NO: 8 - RNA, 31 nt, synthetic construct
  source 1..31: mol_type = other RNA
  modified_base order(3,15,17,19,21,23,25,27,30): mod_base = OTHER; note = Locked nucleic acids
  modified_base order(1..2,4..14,16,18,20,22,24,26,28..29,31): mod_base = OTHER; note = 2-Prime-O-methyl nucleotides
  modified_base order(15,23,27): mod_base = OTHER; note = Thymine
ttacctttcagtgttaagcagcttcttgtcc

SEQ ID NO: 9 - RNA, 21 nt, synthetic construct
  source 1..21: mol_type = other RNA
tcctcactataatccagcaac

SEQ ID NO: 10 - RNA, 21 nt, synthetic construct
  source 1..21: mol_type = other RNA
tgctggattatagtgaggctt

SEQ ID NO: 11 - RNA, 23 nt, synthetic construct
  source 1..23: mol_type = other RNA
  modified_base order(1,4,7,11,14,17,20,23): mod_base = OTHER; note = Locked nucleic acids
  modified_base order(2..3,5..6,8..10,12..13,15..16,18..19,21..22): mod_base = OTHER; note = 2-Prime-O-methyl nucleotides
  modified_base order(4,14,17): mod_base = OTHER; note = Thymine
acattccactgtcttctctggac

SEQ ID NO: 12 - RNA, 23 nt, synthetic construct
  source 1..23: mol_type = other RNA
  modified_base order(1,3,5,7,11,14,17,20,23): mod_base = OTHER; note = Locked nucleic acids
  modified_base order(2,4,6,8..10,12..13,15..16,18..19,21..22): mod_base = OTHER; note = 2-Prime-O-methyl nucleotides
  modified_base order(7,14,17): mod_base = OTHER; note = Thymine
ataagctactgtcttctctggac

SEQ ID NO: 13 - RNA, 23 nt, synthetic construct
  source 1..23: mol_type = other RNA
  modified_base order(1,3,5,7,9,11,13,15,17,19,22): mod_base = OTHER; note = Locked nucleic acids
  modified_base order(2,4,6,8,10,12,14,16,18,20..21,23): mod_base = OTHER; note = 2-Prime-O-methyl nucleotides
  modified_base 21..23: mod_base = OTHER; note = Phosphorothioate internucleotide linkage
  modified_base order(5,15,19): mod_base = OTHER; note = Thymine
acactccaagcagcttcttgtcc

SEQ ID NO: 14 - RNA, 31 nt, synthetic construct
  source 1..31: mol_type = other RNA
  modified_base order(1,3,5,9,11,13,15,17,19,21,23,25,27,30): mod_base = OTHER; note = Locked nucleic acids
  modified_base order(2,4,6..8,10,12,14,16,18,20,22,24,26,28..29,31): mod_base = OTHER; note = 2-Prime-O-methyl nucleotides
  modified_base 1..3: mod_base = OTHER; note = Phosphorothioate internucleotide linkage
  modified_base 29..31: mod_base = OTHER; note = Phosphorothioate internucleotide linkage
  modified_base order(5,13,23,27): mod_base = OTHER; note = Thymine
ccattagaacactccaagcagcttcttgtcc

SEQ ID NO: 15 - RNA, 47 nt, synthetic construct
  source 1..47: mol_type = other RNA
  modified_base 1..47: mod_base = OTHER; note = 2-Prime-O-methyl nucleotides
ccattagaacactccaccattagaacactccaagcttcttgtccagc

SEQ ID NO: 16 - RNA, 20 nt, synthetic construct
  source 1..20: mol_type = other RNA
  modified_base order(1..6,16..20): mod_base = OTHER; note = 2-Prime-O-methyl nucleotides
  modified_base order(7..8,10): mod_base = OTHER; note = Thymine
agcttcttgtccagctttat

SEQ ID NO: 17 - RNA, 31 nt, synthetic construct
  source 1..31: mol_type = other RNA
  modified_base order(1,3,5,9,11,13,15): mod_base = OTHER; note = Locked nucleic acids
  modified_base order(2,4,6..8,10,12,14,16..31): mod_base = OTHER; note = 2-Prime-O-methyl nucleotides
  modified_base 1..3: mod_base = OTHER; note = Phosphorothioate internucleotide linkage
  modified_base 29..31: mod_base = OTHER; note = Phosphorothioate internucleotide linkage
  modified_base order(5,13): mod_base = OTHER; note = Thymine
ccattagaacactccaccggtgttagctttg

SEQ ID NO: 18 - RNA, 47 nt, synthetic construct; identical to SEQ ID NO: 15
  source 1..47: mol_type = other RNA
  modified_base 1..47: mod_base = OTHER; note = 2-Prime-O-methyl nucleotides
  modified_base 1..19: mod_base = OTHER; note = Phosphorothioate internucleotide linkage
  modified_base 44..47: mod_base = OTHER; note = Phosphorothioate internucleotide linkage

SEQ ID NO: 19 - RNA, 47 nt, synthetic construct; identical to SEQ ID NO: 15
  source 1..47: mol_type = other RNA
  modified_base 1..47: mod_base = OTHER; note = 2-Prime-O-methyl nucleotides
  modified_base 1..4: mod_base = OTHER; note = Phosphorothioate internucleotide linkage
  modified_base 29..47: mod_base = OTHER; note = Phosphorothioate internucleotide linkage

SEQ ID NO: 20 - RNA, 47 nt, synthetic construct; identical to SEQ ID NO: 15
  source 1..47: mol_type = other RNA
  modified_base 1..47: mod_base = OTHER; note = 2-Prime-O-methyl nucleotides
  modified_base 1..11: mod_base = OTHER; note = Phosphorothioate internucleotide linkage
  modified_base 44..47: mod_base = OTHER; note = Phosphorothioate internucleotide linkage

SEQ ID NO: 21 - RNA, 47 nt, synthetic construct; identical to SEQ ID NO: 15
  source 1..47: mol_type = other RNA
  modified_base 1..47: mod_base = OTHER; note = 2-Prime-O-methyl nucleotides
  modified_base 1..4: mod_base = OTHER; note = Phosphorothioate internucleotide linkage
  modified_base 37..47: mod_base = OTHER; note = Phosphorothioate internucleotide linkage

SEQ ID NO: 22 - RNA, 22 nt, synthetic construct
  source 1..22: mol_type = other RNA
tggaatgtaaagaagtatgtat

SEQ ID NO: 23 - RNA, 22 nt, synthetic construct
  source 1..22: mol_type = other RNA
tggagtgtgacaatggtgtttg

SEQ ID NO: 24 - RNA, 25 nt, synthetic construct
  source 1..25: mol_type = other RNA
  modified_base order(1,4,7,11,14,17,20,23): mod_base = OTHER; note = Locked nucleic acids
  modified_base order(2..3,5..6,8..10,12..13,15..16,18..19,21..22): mod_base = OTHER; note = 2-Prime-O-methyl nucleotides
  modified_base 1..4: mod_base = OTHER; note = Phosphorothioate internucleotide linkage
  modified_base 20..23: mod_base = OTHER; note = Phosphorothioate internucleotide linkage
  modified_base order(4,14,17): mod_base = OTHER; note = Thymine
  misc_feature 24..25: note = dTdT dinucleotide linker
  modified_base 24..25: mod_base = OTHER; note = Deoxythymidine
acattccactgtcttctctggactt

SEQ ID NO: 25 - RNA, 25 nt, synthetic construct
  source 1..25: mol_type = other RNA
  modified_base order(1,3,5,7,11,14,17,20,23): mod_base = OTHER; note = Locked nucleic acids
  modified_base order(2,4,6,8..10,12..13,15..16,18..19,21..22): mod_base = OTHER; note = 2-Prime-O-methyl nucleotides
  modified_base 1..4: mod_base = OTHER; note = Phosphorothioate internucleotide linkage
  modified_base 20..23: mod_base = OTHER; note = Phosphorothioate internucleotide linkage
  modified_base order(3,5,14,17): mod_base = OTHER; note = Thymine
  misc_feature 24..25: note = dTdT dinucleotide linker
  modified_base 24..25: mod_base = OTHER; note = Deoxythymidine
cgtcttatctgtcttctctggactt

(19) United States
(12) Patent Application Publication
(10) Pub. No.: US 2023/0399644 A1
(43) Pub. Date: Dec. 14, 2023

(54) SELECTIVE RNA-MODULATING AGENTS

(71) Applicant: UNIVERSITY OF MASSACHUSETTS, Boston, MA (US)

(72) Inventors: Phillip D. Zamore, Northboro, MA (US); Jennifer A. Broderick, Worcester, MA (US)

(21) Appl. No.: 18/140,908

(22) Filed: Apr. 28, 2023

Related U.S. Application Data
(60) Provisional application No. 63/354,435, filed on Jun. 22, 2022.
(60) Provisional application No. 63/336,585, filed on Apr. 29, 2022.

(51) Int. Cl. C12N 15/113; A61P 21/00

(52) U.S. Cl. CPC C12N 15/113; A61P 21/00; C12N 2310/3231; C12N 2310/141

(57) ABSTRACT

The instant disclosure provides RNA-modulating agents that function to recruit one or more small regulatory RNA molecules (e.g., miRNA molecules, Y RNAs, and siRNAs) to a target mRNA thereby modulating (e.g., inhibiting) the translation of the target mRNA or destabilizing the mRNA. Methods for using the RNA-modulating agents are also provided.

### CROSS REFERENCE TO RELATED APPLICATION

This application claims the benefit of U.S. Provisional Patent Application Ser. No. 63/336,585, filed Apr. 29, 2022, and U.S. Provisional Patent Application Ser. No. 63/354,435, filed Jun. 22, 2022. The entire contents of the above-referenced patent applications are incorporated by reference in their entirety herein.

### SEQUENCE LISTING

The instant application contains a Sequence Listing which has been submitted electronically in XML format and is hereby incorporated by reference in its entirety. Said XML file, created on Aug. 21, 2023, is named 740074_UM9-281_ST26.xml and is 62,331 bytes in size.

### FIELD OF THE INVENTION

This disclosure relates to compositions and methods of using RNA-modulating agents.

### BACKGROUND

Small interfering RNAs (siRNAs) and microRNAs (miRNAs) repress gene expression through nucleic acid base-pairing between the target mRNA and the small RNA guide bound to a member of the Argonaute family of proteins. The use of siRNAs and miRNAs to treat diseases and viral infections of non-hepatic origin requires designing siRNAs and miRNAs that effectively trigger gene silencing in vivo, resist nucleolytic degradation, and accumulate in the correct tissue and cell type. Presently, no current delivery strategy can effectively and selectively silence disease-causing or viral genes in some tissues but not others.

Accordingly, there is a need in the art for novel compositions for mediating gene silencing that can be administered systemically, but yet can act in a tissue specific manner.

### SUMMARY

The instant disclosure provides RNA-modulating agents that function to recruit one or more miRNA molecules to a target mRNA thereby modulating (e.g., inhibiting) the translation of the target mRNA or destabilizing the mRNA. The instant RNA-modulating agents comprise an mRNA binding sequence that is complementary to a portion of a target mRNA sequence, linked to one or more miRNA binding sequences, wherein the one or more miRNA binding sequences are complementary to at least positions 2 to 8 of a miRNA, and wherein the miRNA binding sequence comprises at least one modified nucleotide at nucleotide positions that are complementary to at one or more of positions 2, 5, and 8 of the miRNA.

The instant disclosure is based, in part, on the discovery that incorporating the modification at the nucleotides that pair with positions 2, 5 and 8 of the microRNA (e.g., miR-1) allowed the RNA-modulating agent to silence the target mRNA when the microRNA binding site contained only nucleotides that pair to the seed (nucleotide positions 1-8) and no supplemental pairing at positions 12-15. This shortened the total length of the tether to 23 nucleotides. With the new modifications, the tethers are now nearly the length of one strand of an siRNA (21 nucleotides). These chemical modifications allow the RNA-modulating agent to distinguish between the seed sequence of microRNAs that differ by as little as one nucleotide (e.g., miR-1 and miR-122, which differ only at position 5).

The instant disclosure is also based on the discovery that the RNA-modulating agents can achieve tissue specific silencing with systemic administration. The highly selective RNA-modulating agents will only function in cells that contain the target miRNA and the target mRNA, in particular where said miRNA is sufficiently abundant to mediate modulation of the target mRNA.

Accordingly, in one aspect the instant disclosure provides an RNA-modulating agent comprising an mRNA binding sequence that is complementary to a portion of a target mRNA sequence, linked to one or more miRNA binding sequences,
wherein the one or more miRNA binding sequences are complementary to at least positions 2 to 8 of a miRNA from the 5′ end of the miRNA, and
wherein the miRNA binding sequence comprises at least one modified nucleotide at a nucleotide position that is complementary to one or more of positions 2, 5, and 8 of the miRNA from the 5′ end of the miRNA.

In certain embodiments, the miRNA binding sequence comprises a modified nucleotide at nucleotide positions that are complementary to positions 2, 5, and 8 of the miRNA.

In certain embodiments, the modified nucleotide is selected from a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide.

In certain embodiments, the modified nucleotide comprises an LNA or a PNA.

In certain embodiments, the RNA-modulating agent reduces target mRNA abundance by at least 50% in a tissue.

In certain embodiments, the RNA-modulating agent is capable of discriminatory binding of the target miRNA relative to a non-target miRNA.

In certain embodiments, the non-target miRNA sequence differs from the target miRNA sequence by at least one nucleotide (e.g., one nucleotide, two nucleotides, or three nucleotides).

In certain embodiments, the RNA-modulating agent comprises one miRNA binding sequence. In certain embodiments, the RNA-modulating agent comprises two or more miRNA binding sequences.

In certain embodiments, the target mRNA is a neuromuscular mRNA target.

In certain embodiments, the target miRNA is a miRNA expressed in muscle tissue. In certain embodiments, the muscle tissue is skeletal muscle and/or cardiac muscle. In certain embodiments, the target miRNA is a miRNA expressed in neuronal tissue.

In certain embodiments, the target miRNA is a miRNA expressed in a specific tissue within a multicellular organism. In certain embodiments, the multicellular organism is a mammal. In certain embodiments, the multicellular organism is a plant.

In certain embodiments, the miRNA exhibits a tissue specific expression pattern.

In certain embodiments, the one or more miRNA binding sequences are not complementary to positions 10 and 11 of the miRNA.

In certain embodiments, the one or more miRNA binding sequences are complementary to positions 2 to 8, and 12 to 15, 12 to 16, or 12 to 17 of the miRNA, but not complementary to positions 10 and 11 of the miRNA.

In certain embodiments, the one or more miRNA binding sequences are not complementary to position 1 of the miRNA.

In certain embodiments, the one or more miRNA binding sequences are complementary to positions 2 to 8, and 12 to 15, 12 to 16, or 12 to 17 of the miRNA, but not complementary to positions 1, 10 and 11 of the miRNA.

In certain embodiments, the miRNA binding sequences are complementary to only positions 2 to 8 of the miRNA.

In certain embodiments, the one or more miRNA binding sequences have an adenosine, at a position in the miRNA binding sequence corresponding to position 9 of the miRNA.

In certain embodiments, the one or more miRNA binding sequences are about 8 to about 25 nucleotides in length (e.g., 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides).

In certain embodiments, the one or more miRNA binding sequences are 8 nucleotides in length.

In certain embodiments, the mRNA binding sequence is about 15 nucleotides in length.

In certain embodiments, the target miRNA is selected from the group consisting of miR-1, miR-24, miR-32, miR-103, miR-107, miR122, miR-124, miR-125, miR-127, miR-128, miR-130, miR-132, miR-134, miR-135, miR-138, miR-143, miR-148, miR-150, miR-151, miR-152, miR-153, miR-181, miR-189, miR-192, miR-194, miR-195, miR-199, miR-203, miR-204, miR-206, miR-208, miR-212, miR-215, miR-216, miR-221, miR-222, miR-375, miR-378, miR-30b, miR-30c, miR-122a, miR-133a, miR-200a, miR-142-3p, miR-143-5p, let-7, and a viral microRNA.

In certain embodiments, the target miRNA is miR-1. In certain embodiments, the target miRNA is miR-122. In certain embodiments, the target miRNA is miR-192. In certain embodiments, the target miRNA is miR-203. In certain embodiments, the target miRNA is miR-208.

In certain embodiments, a functional moiety is linked to the 5′ end and/or 3′ end of the RNA-modulating agent. In certain embodiments, a functional moiety is linked to the 3′ end of the RNA-modulating agent.

In certain embodiments, the functional moiety comprises a hydrophobic moiety.

In certain embodiments, the hydrophobic moiety is selected from the group consisting of fatty acids, steroids, secosteroids, lipids, gangliosides, nucleoside analogs, endocannabinoids, vitamins, and a mixture thereof.

In certain embodiments, the steroid selected from the group consisting of cholesterol and Lithocholic acid (LCA).

In certain embodiments, the fatty acid selected from the group consisting of Eicosapentaenoic acid (EPA), Docosahexaenoic acid (DHA) and Docosanoic acid (DCA).

In certain embodiments, the vitamin is selected from the group consisting of choline, vitamin A, vitamin E, and derivatives or metabolites thereof.

In certain embodiments, the vitamin is selected from the group consisting of retinoic acid and alpha-tocopheryl succinate.

In certain embodiments, the functional moiety is linked to the RNA-modulating agent by a linker.

In certain embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments, the linker is a cleavable linker.

In certain embodiments, the linker comprises a dTdT dinucleotide.

In certain embodiments, the target mRNA is expressed in skeletal muscle and the target miRNA is miR-1. In certain embodiments, the target mRNA is activin A.

In certain embodiments, the mRNA binding sequence comprises CUGUCUUCUCUGGAC (SEQ ID NO: 1).

In certain embodiments, the one or more miRNA binding sequences comprises ACAUUCCA.

In certain embodiments, the target mRNA is expressed in cardiac muscle and the target miRNA is miR-208. In certain embodiments, the target mRNA is MARK4, VASH1, or VASH2.

In certain embodiments, the target mRNA is expressed in liver and the target miRNA is miR-122. In certain embodiments, the target mRNA is ApoC3.

In certain embodiments, the target mRNA is expressed in kidney and the target miRNA is miR-192. In certain embodiments, the target mRNA is Smad3.

In certain embodiments, the target mRNA is expressed in skin and the target miRNA is miR-203. In certain embodiments, the target mRNA is elastase, cathepsin K, or a matrix metallo-proteinase (MMP) mRNA (e.g., MMP-1, MMP-8, MMP-13, MMP-14, MMP-16, and MMP-18).

In certain embodiments, the RNA-modulating agent comprises a chemical modification pattern of
(lN)#(mN)#(mN)#(lN)(mN)(mN)(lN)(mN)(mN)(mN)(lN)(mN)(mN)(lN)(mN)(mN)(lN)(mN)(mN)(lN)#(mN)#(mN)#(lN),
   wherein “l” corresponds to an LNA modification, “m” corresponds to a 2′-O-methyl modification, “#” corresponds to a phosphorothioate internucleotide linkage, and “N” corresponds to any nucleotide (A, T, U, G, or C).

In one aspect, the disclosure provides a method of treating a subject having a disease or disorder characterized by or caused by:
(a) the overexpression or overactivity of a normal cellular protein;
(b) the underexpression or underactivity of a normal cellular protein;
(c) the activity of a mutant protein; or
(d) the activity of a viral RNA or protein,
the method comprising administering to the subject an effective amount of an RNA-modulating agent described above, wherein the RNA-modulating agent binds to the mRNA encoding the protein and modulates expression of a protein.

In one aspect, the disclosure provides a method of modulating the expression of a neuromuscular target mRNA in a subject, the method comprising administering to the subject an effective amount of an RNA-modulating agent, wherein the RNA-modulating agent comprises an mRNA binding sequence that is complementary to a portion of the neuromuscular target mRNA, linked to one or more miRNA binding sequences,
wherein the one or more miRNA binding sequences are complementary to at least positions 2 to 8 of a miRNA, and
wherein the RNA-modulating agent binds to the neuromuscular target mRNA, thereby modulating the expression of the neuromuscular target mRNA.

In one aspect, the disclosure provides a method of modulating the expression of a target mRNA in skeletal muscle in a subject, the method comprising administering to the subject an effective amount of an RNA-modulating agent,
wherein the RNA-modulating agent comprises an mRNA binding sequence that is complementary to a portion of the target mRNA, linked to one or more miR-1 binding sequences,
wherein the one or more miRNA binding sequences are complementary to at least positions 2 to 8 of miR-1, and
wherein the RNA-modulating agent binds to the target mRNA, thereby modulating the expression of the target mRNA in the skeletal muscle in the subject.

In one aspect, the disclosure provides a method of modulating the expression of a target mRNA in cardiac muscle in a subject, the method comprising administering to the subject an effective amount of an RNA-modulating agent,
wherein the RNA-modulating agent comprises an mRNA binding sequence that is complementary to a portion of the target mRNA, linked to one or more miR-208 binding sequences,
wherein the one or more miRNA binding sequences are complementary to at least positions 2 to 8 of miR-208, and
wherein the RNA-modulating agent binds to the target mRNA, thereby modulating the expression of the target mRNA in the cardiac muscle in the subject.

In one aspect, the disclosure provides a method of modulating the expression of a target mRNA in kidney in a subject, the method comprising administering to the subject an effective amount of an RNA-modulating agent,
wherein the RNA-modulating agent comprises an mRNA binding sequence that is complementary to a portion of the target mRNA, linked to one or more miR-192 binding sequences,
wherein the one or more miRNA binding sequences are complementary to at least positions 2 to 8 of miR-192, and
wherein the RNA-modulating agent binds to the target mRNA, thereby modulating the expression of the target mRNA in the kidney in the subject.

In one aspect, the disclosure provides a method of modulating the expression of a target mRNA in skin in a subject, the method comprising administering to the subject an effective amount of an RNA-modulating agent,
wherein the RNA-modulating agent comprises an mRNA binding sequence that is complementary to a portion of the target mRNA, linked to one or more miR-203 binding sequences,
wherein the one or more miRNA binding sequences are complementary to at least positions 2 to 8 of miR-203, and
wherein the RNA-modulating agent binds to the target mRNA, thereby modulating the expression of the target mRNA in the skin in the subject.

In one aspect, the disclosure provides a method of modulating the expression of a target mRNA in liver in a subject, the method comprising administering to the subject an effective amount of an RNA-modulating agent,
wherein the RNA-modulating agent comprises an mRNA binding sequence that is complementary to a portion of the target mRNA, linked to one or more miR-122 binding sequences,
wherein the one or more miRNA binding sequences are complementary to at least positions 2 to 8 of miR-122, and
wherein the RNA-modulating agent binds to the target mRNA, thereby modulating the expression of the target mRNA in the liver in the subject.

In certain embodiments of the methods above, the miRNA binding sequence comprises at least one modified nucleotide at one or more of positions 2, 5, and 8 of the miRNA.

In certain embodiments of the methods above, the miRNA binding sequence comprises a modified nucleotide at least at positions 2, 5, and 8 of the miRNA.

In certain embodiments of the methods above, the modified nucleotide is selected from a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide.

In certain embodiments of the methods above, the modified nucleotide comprises an LNA or a PNA.

In certain embodiments of the methods above, the RNA-modulating agent is capable of discriminatory binding of the target miRNA relative to a non-target miRNA.

In certain embodiments of the methods above, the non-target miRNA sequence differs from the target miRNA sequence by at least one nucleotide.

In certain embodiments of the methods above, the RNA-modulating agent comprises one miRNA binding sequence.

In certain embodiments of the methods above, the RNA-modulating agent comprises two or more miRNA binding sequences.

In another aspect, the disclosure provides a method of treating or preventing sarcopenia and/or cachexia in a subject, the method comprising administering to the subject an effective amount of an RNA-modulating agent,
wherein the RNA-modulating agent comprises an mRNA binding sequence that is complementary to a portion of an activin A mRNA, linked to a miRNA binding sequence,
wherein the miRNA binding sequence is complementary to at least positions 2 to 8 of a target miRNA, and
wherein the RNA-modulating agent binds to the activin A mRNA, thereby treating or preventing sarcopenia and/or cachexia in the subject.

In certain embodiments of the method above, the miRNA binding sequence comprises at least one modified nucleotide at one or more of positions 2, 5, and 8 of the miRNA.

In certain embodiments of the method above, the target miRNA is miR-1.

In certain embodiments of the method above, the miRNA binding sequence comprises, from 5′ to 3′, ACAUUCCA.

In certain embodiments of the method above, the mRNA binding sequence comprises, from 5′ to 3′, CUGUCUUCUCUGGAC (SEQ ID NO: 1).

In certain embodiments of the method above, the RNA-modulating agent comprises, from 5′ to 3′, ACAUUCCACUGUCUUCUCUGGAC (SEQ ID NO:2).

In certain embodiments of the method above, the RNA-modulating agent comprises, from 5′ to 3′,
(lA)#(mC)#(mA)#(lT)(mU)(mC)(lC)(mA)(mC)(mU)(lG)(mU)(mC)(lT)(mU)(mC)(lT) (mC)(mU)(lG)#(mG)#(mA)#(lC) (SEQ ID NO:3),
   wherein “l” corresponds to an LNA modification, “m” corresponds to a 2′-O-methyl modification, and “#” corresponds to a phosphorothioate internucleotide linkage.

In one aspect, the disclosure provides an RNA-modulating agent comprising an mRNA binding sequence that is complementary to a portion of an Activin A mRNA sequence, linked to one or more miRNA binding sequences.

In certain embodiments, the one or more miRNA binding sequences is complementary to at least positions 2 to 8 of a miRNA from the 5′ end of the miRNA.

In certain embodiments, the miRNA binding sequence comprises at least one modified nucleotide at a nucleotide position that is complementary to any one or more of positions 2, 5, and 8 of the miRNA from the 5′ end. of the miRNA.

In certain embodiments, the miRNA binding sequence comprises at least one modified nucleotide at one or more of positions 2, 5, and 8 of the miRNA.

In certain embodiments, the target miRNA is miR-1.

In certain embodiments, the miRNA binding sequence comprises, from 5′ to 3′, ACAUUCCA.

In certain embodiments, the mRNA binding sequence comprises, from 5′ to 3′,

(SEQ ID NO: 1)
CUGUCUUCUCUGGAC.

In certain embodiments, the RNA-modulating agent comprises, from 5′ to 3′,

(SEQ ID NO: 2)
ACAUUCCACUGUCUUCUCUGGAC.

In certain embodiments, the RNA-modulating agent comprises at least one modified nucleotide at nucleotide.

In certain embodiments, the miRNA binding sequence comprises at least one modified nucleotide.

In certain embodiments, the miRNA binding sequence comprises a modified nucleotide at nucleotide positions that are complementary to positions 2, 5, and 8 of the miRNA.

In certain embodiments, the modified nucleotide is selected from a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide.

In certain embodiments, the modified nucleotide comprises an LNA or a PNA.

In certain embodiments, the RNA-modulating agent comprises, from 5′ to 3′,
(lA)#(mC)#(mA)#(lT)(mU)(mC)(lC)(mA)(mC)(mU)(lG)(mU)(mC)(lT)(mU)(mC)(lT) (mC)(mU)(lG)#(mG)#(mA)#(lC) (SEQ ID NO:3),
   wherein “l” corresponds to an LNA modification, “m” corresponds to a 2′-O-methyl modification, and “#” corresponds to a phosphorothioate internucleotide linkage.

### BRIEF DESCRIPTION OF THE FIGURES

FIG. 1 depicts activin A mRNA levels normalized to GAPDH in mouse myotubes incubated with an RNA-modulating agent (‘tether”). C2C12 mouse myotubes were transfected with 20 nM of an RNA-modulating agent targeting activin A mRNA for silencing using different miRNAs. Abundance of Activin A mRNA was measured by qRT-PCR 72 h post transfection and normalized to GAPDH and control tether. Data was normalized to transfection efficiency based on percent of cells expressing yellow fluorescent protein. Data is the mean±standard deviation from three independent replicates.

FIG. 2 depicts activin A mRNA levels normalized to GAPDH in C2C12 mouse myotubes (top bar) and Huh7.5 human hepatocytes (bottom bar) incubated with an RNA-modulating agent. Cells were transfected with 20 nM of each RNA-modulating agent. Abundance of Activin A mRNA was measured by qRT-PCR 72 h post transfection and normalized to GAPDH and control tether. Data is the mean±standard error from three independent replicate. Tethers without LNA at the nucleotide that pairs with position 5 of the seed sequence of miR-1 silenced the target in both cell types, suggesting that the tether binds miR-122 in hepatocytes that do not express miR-1. Tethers with LNA at the nucleotide that pairs with position 5 of the seed sequence of miR-1 silenced the target in myotubes that express miR-1, but not in heptocytes.

FIG. 3 depicts in vivo administration of RNA-modulating agents in mice. Female C57bl/6 mice, aged 23 months, received 4 interscapular subcutaneous doses of 10 mg/kg of tether A or tether B. 28 days post-injection the mass of each quadriceps muscle was measured and normalized to body weight. The group (n=20) that received tether A had a significantly increased sarcopenic index for quadriceps mass (mg)/body weight (g) of 5.2 (p=0.017, one-way ANOVA, Dunnett's multiple comparisons test) compared to 4.6 in the control group (n=23). The tether B group (n=24) had a sarcopenic index of 5.0 (p=0.106). Black bar indicates median.

FIGS. 4A-C depict Activin A mRNA level in quadriceps lysate (FIG. 4A), heart lysate (FIG. 4B), and liver lysate (FIG. 4C) after miR-1 and mirR-208 injections. A miR-1 tether significantly decreased activin A mRNA in quadriceps muscle lysate. The miR-208 tether did not reduce activin A in quadriceps lysate because it should only silence activin A in heart tissue. Activin A mRNA was not significantly decreased in heart lysate after 28 days. A 3-month study may detect a change in activin A levels in heart tissue. Due to liver exposure of the DCA-conjugated tethers, the level of activin A mRNA in liver lysate was assess but significant Activin A silencing in liver was not detected. One-way ANOVA, Tukey's multiple comparisons adjusted p value.

FIGS. 5A-F depict mice gastrocnemius mass relative level (FIG. 5A), tibialis anterior mass relative level (FIG. 5B), heart mass relative level (FIG. 5C), and body weight over time (FIGS. 5D-F) for miR-1 and miR-208 injections.

FIG. 6 (SEQ ID NO: 22 AND 23) depicts locked nucleic acid (LNA) modified RNA-modulating agent discrimination between miRNAs with near cognate seed sequences.

FIG. 7 depicts a tether recruiting miR-122 RISC to a single 15 nt site in 3′ UTR of ApoC-III.

FIG. 8 depicts three oligonucleotide tethers and a tether complementary to miR-200a. The three oligonucleotide tethers were designed to recruit miR-122 to ApoC3 mRNA and were found to reduce ApoC3 mRNA abundance in hepatocyte-derived Huh7.5 cells. In contrast, the tether complementary to miR-200a is a miRNA not expressed in Huh7.5 cells and was found to do not decrease ApoC3 mRNA abundance in hepatocyte-derived Huh7.5 cells. Huh7.5 cells were co-transfected with 20 nM tether oligonucleotides and 1 μg pEYFP plasmid (TransIT-X2, MirusBio). RNA was harvested (RNAeasy Plus, Qiagen) 72 h later. Transfection efficiency was determined in parallel by measuring the percent of cells expressing YFP (MACSQuant® VYB, Miltenyi Biotech). ApoC3 and GAPDH mRNA levels were measured by RT-qPCR. Bars represent the mean±SEM (n≥3).

FIG. 9 depicts an oligonucleotide tether designed to bind miR-122 and APOC3 mRNA. The oligonucleotide significantly was found to reduce serum triglycerides in vivo in mice. The 3′ end of the tether (tether design #3) was conjugated to GalNAc to facilitate delivery to hepatocytes. Mice received 3.3 mg per kg tether by interscapular sub-cutaneous injection on days 1, 2, and 3. Serum was collected 10 days before the first injection (baseline) and 7 days after the last injection. Statistical significance was measured with a paired, two-tailed Student's t-test. Horizontal bars: mean±SEM.

FIG. 10 depicts the activity of ApoC3 tethers to reduce ApoC3 mRNA in mouse liver. Tethers with 10 or 18 phosphorothioate linkages in the 5′ end of the tether reduced mRNA levels by 50% (blue and red arrowheads). Tethers with 10 or 18 phosphorothioate linkages in the 3′ end of the tether, next to the GalNAc, did not reduce mRNA levels (n=5 C57Bl/6 mice per group). The 3′ end of the tether (tether design #3) was conjugated to GalNAc to facilitate delivery to hepatocytes. Mice received a single 10 mg per kg tether by interscapular sub-cutaneous injection on day 1. The amount of tethers and ApoC3 mRNA in liver tissue was measured by Quantigene Assay after 48 hours. Circles indicate amount of tether detected in liver lysate (micrograms of tether per gram of liver, color of circle corresponds to the tether received, red, 1447; blue, 1449; green, 1448; black, 1450; grey, PBS. Statistical significance was measured with an unpaired, two-tailed Student's t-test.

### DETAILED DESCRIPTION

The instant disclosure provides RNA-modulating agents that function to recruit one or more miRNA molecules to a target mRNA thereby modulating (e.g., inhibiting) the translation of the target mRNA or destabilizing the mRNA. Methods for using the RNA-modulating agents are also provided.

Endogenous miRNAs are among the most tissue-specific regulators of gene expression. For example, the miRNA miR-1 is largely if not entirely restricted to muscle, while miR-122 has been found only in liver. We have developed anew approach to gene silencing that combines the tissue-specificity and innate silencing capacity of endogenous miRNAs with the well-established stability and broad deliverability of antisense oligonucleotides. The strategy employed herein uses an oligonucleotide tether (i.e., RNA modulating agent) to recruit an endogenous miRNA to a specific target mRNA. The tether combines a sequence complementary to the target mRNA with a second sequence complementary to an abundant endogenous miRNA found in the tissue of interest. The tether binds endogenous miRNA-loaded Argonaute complexes and recruits them to the mRNA. MicroRNA tethering is the only therapeutic strategy that limits silencing to the intended cell type even when the therapeutic agent is delivered systemically to many tissues. It is an area of unmet medical need—a way to selectively target disease tissue and spare normal tissue.

The RNA-modulating agents described herein have the ability to exploit the cell-type specific expression of miRNAs. The RNA-modulating agents silence in one cell type and not another, dependent upon the level of miRNAs in each cell type. The amount of a miRNA in a cell should be an amount sufficient for tethers to silence the target gene. For example, in a tissue that expresses 500 copies per cell of a miRNA, a tether may not silence the target gene. However, in an adjacent tissue or tumor, for example there may be 10,000 or even >100,000 copies per cell of the same miRNA; such high levels would allow the tethers to selectively silence the disease gene in the target tissue or cell type. Such cell-type specificity is unique to tethers and could allow for safer than standard-of-care medicines with dose-limiting toxicities and collateral tissue damage.

### I. Definitions

As used herein, the term “RNA-modulating agent” or “oligonucleotide tether” refers to a molecule comprising an mRNA binding sequence and at least one miRNA binding sequence. Non-limiting examples of RNA-modulating agents are set forth in US20050256072, US20060293267, and US20160089453, which are both incorporated herein by reference in their entirety.

As used herein, the term “mRNA binding sequence” refers to an oligonucleotide, or mimetic thereof, having a nucleotide sequence that is complementary to the nucleotide sequence of an mRNA.

As used herein, the term “miRNA binding sequence” refers to an oligoribonucleotide, or analogue thereof, having a nucleotide sequence that is complementary to the nucleotide sequence of an miRNA.

As used herein, the terms “microRNA” or “miRNA” refer to the class of naturally occurring, small, non-coding RNA molecules, about 21-25 nucleotides in length, that function to modulate gene expression in a variety of ways, including translational repression, mRNA cleavage, and deadenylation. The complete listing of published miRNA sequences as are set forth at mirbase.org.

As used herein, the term “complementary” refers to the ability of nucleotides, or analogues thereof, to form Watson-Crick base pairs. Complementary nucleotide sequences will form Watson-Crick base pairs and non-complementary nucleotide sequences will not.

As used herein, the term “position in the miRNA binding sequence corresponding to position 9 of the miRNA” refers to the nucleotide in an miRNA binding sequence that, in a perfect duplex of an miRNA binding sequence and its cognate miRNA, would form Watson-Crick base pairs with the 9th nucleotide of the miRNA (the miRNA being numbered from 5′ to 3′).

As used herein, the term “oligonucleotide” refers to a polymer of nucleotides comprising naturally occurring nucleotides, non-naturally occurring nucleotides, derivatized nucleotides, or a combination thereof. Non-limiting examples of nucleotides, and derivatives thereof, are set forth herein.

As used herein, the term “oligoribonucleotide” refers to a polymer of nucleotides comprising naturally occurring ribonucleotides, non-naturally occurring ribonucleotides, derivatized ribonucleotides, or a combination thereof. Non-limiting examples of ribonucleotides, and derivatives thereof, are set forth herein.

The term “RNA” or “RNA molecule” or “ribonucleic acid molecule” refers to a polymer of ribonucleotides. The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains.

### II. RNA-Modulating Agents

The instant disclosure provides an RNA-modulating agent comprising an mRNA binding sequence that is complementary to a portion of a target mRNA sequence, linked to one or more miRNA binding sequences, wherein the one or more miRNA binding sequences is complementary to at least positions 2 to 8 of a miRNA, and wherein the miRNA binding sequence comprises at least one modified nucleotide at nucleotide positions that are complementary to at one or more of positions 2, 5, and 8 of the miRNA.

In certain embodiments, the miRNA binding sequence comprises a modified nucleotide at nucleotide positions that are complementary to positions 2, 5, and 8 of the miRNA.

In certain embodiments, the modified nucleotide is selected from a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide.

In certain embodiments, the modified nucleotide comprises an LNA or a PNA. In particular, the miRNA binding sequence comprises an LNA or PNA modification at nucleotide positions that are complementary to each of positions 2, 5, and 8 of the miRNA.

The RNA-modulating agents of the disclosure are capable of discriminatory binding of the target miRNA relative to a non-target miRNA. The non-target miRNA sequence differs from the target miRNA sequence by at least one nucleotide (e.g., 1 nucleotide, 2 nucleotides, or 3 nucleotides).

The miRNA binding sequences disclosed herein are complementary to at least positions 2 to 8 of the miRNA, optionally with complementarity to other positions of the miRNA. In certain embodiments, the miRNA binding sequence is not complementary to positions 10 and 11 of the miRNA. In certain embodiments, the miRNA binding sequence is complementary to positions 2 to 8, and 12 to 15, 12 to 16, or 12 to 17 of the miRNA, but not complementary to positions 10 and 11 of the miRNA. In certain embodiments, the miRNA binding sequence is not complementary to position 1 of the miRNA. In certain embodiments, the miRNA binding sequence is complementary to positions 2 to 8, and 12 to 15, 12 to 16, or 12 to 17 of the miRNA, but not complementary to positions 1, 10 and 11 of the miRNA. In certain embodiments, the miRNA binding sequence is complementary to positions 1 to 8 of the miRNA. In certain embodiments, the miRNA binding sequence is complementary to only positions 2 to 8 of the miRNA. In certain embodiments, the miRNA binding sequence has an adenosine at a position in the miRNA binding sequence corresponding to position 9 of the miRNA.

The RNA-modulating agents of the disclosure comprises at least one miRNA binding sequence. In certain embodiments, the RNA-modulating agent comprises or consists of one miRNA binding sequence. In certain embodiments, the RNA-modulating agent comprises two or more miRNA binding sequences (e.g., two or three miRNA binding sequences).

The miRNA binding sequence can be of any length sufficient to recruit the desired miRNA. In certain embodiments, the miRNA binding sequence is about 8 to about 25 nucleotides in length (e.g., 8, 9, 10, 11, 12, 13, 14, 15, 16, 17, 18, 19, 20, 21, 22, 23, 24, or 25 nucleotides in length). In certain embodiments, the miRNA binding sequence is 8 nucleotides in length. In other embodiments, the miRNA binding sequence is 8 nucleotides in length and complementary to positions 1 to 8 of the miRNA or positions 2 to 8 of the miRNA.

The miRNA binding sequence can have any amount of complementarity with its cognate miRNA (e.g., 1, 2, 3, 4, 5, 6, 7, 8, 9, 10, 11, 12, 13, 14, 15, 14, 15, 16, 17, 18, 19, 20, 21, 22, 23, 24, and 25 complementary nucleotides). The miRNA binding sequence can also be engineered to comprise specific combinations of Watson-Crick base pairs and mismatches with its cognate miRNA binding partner.

An RNA-modulating agent can comprise an mRNA binding sequence that is complementary to any portion of a target mRNA. In certain embodiments, the mRNA binding sequence is complementary to the 3′UTR of a target mRNA. The mRNA binding sequence should be of sufficient size to effectively bind the target mRNA. The length of the mRNA binding sequence will vary greatly depending, in part, on the length of the target mRNA and the degree of complementarity between the target mRNA and the mRNA binding sequence. In certain embodiments, the mRNA binding sequence is less than about 200, 100, 50, 30, 25, 20, 19, 18, 17, 16, 15, 14, 13, 12, 11, 10, 9, 8, 7, 6, or 5 nucleotides in length. In a particular embodiment, the mRNA binding sequence is about 15 to about 25 nucleotides in length (e.g., 15, 16, 17, 18, 19, 20, 21, 22, 23, 24, or 25 nucleotides in length).

Accordingly, in certain embodiments, the RNA-modulating agent comprises a mRNA binding sequence that is 15 nucleotides in length and a miRNA binding sequence that is 8 nucleotides in length. In this embodiment, the RNA-modulating agent is 23 nucleotides in length.

Any mRNA can be modulated using the RNA-modulating agents disclosed herein, including nuclear mRNA, cytoplasmic mRNA, mitochondrial mRNA, chloroplast mRNA, and viral RNA. When the target mRNA is a nuclear mRNA or cytoplasmic mRNA, the RNA-modulating agents generally cause gene silencing. However, when the target mRNA is a mitochondrial mRNA, the RNA-modulating agents can be used to increase expression of the encoded protein. In certain embodiments, the target mRNA encodes a protein that is overexpressed or overactive in a cell. In certain embodiments, the target mRNA encodes a protein that causes a disease or disorder in an organism (e.g., a human subject or plant), e.g., Huntington's disease (HD) or Amyotrophic lateral sclerosis (ALS). In certain embodiments, the target mRNA encodes a gain of function mutant protein (e.g., mutant huntingtin or SOD1 proteins). In certain embodiments, the target mRNA encodes a protein that is underexpressed or underactive (e.g., a mitochondrial encoded protein). In certain embodiments, the target mRNA encodes huntingtin, APOC3, or SOD1.

In certain embodiments, the target mRNA is a neuromuscular mRNA target (i.e., an mRNA that is involved in neuromuscular activity).

In certain embodiments, the target mRNA is expressed in skeletal muscle (e.g., activin A mRNA).

In certain embodiments, the target mRNA is expressed in cardiac (e.g., MARK4, VASH1, or VASH2 mRNA).

In certain embodiments, the target mRNA is expressed in liver (e.g., ApoC3 mRNA).

In certain embodiments, the target mRNA is expressed in kidney (e.g., Smad3 mRNA).

In certain embodiments, the target mRNA is expressed in skin (e.g., elastase, cathepsin K, or a matrix metallo-proteinase (MMP) mRNA (e.g., MMP-1, MMP-8, MMP-13, MMP-14, MMP-16, and MMP-18).

Any miRNA can be recruited using the RNA-modulating agents disclosed herein. Recruited miRNA can be naturally occurring, viral or synthetic. In certain embodiments, the miRNA is a tissue or cell type specific miRNA or viral miRNA. Recruitment of such miRNA allows for tissue specific or cell type specific gene modulation (e.g., gene silencing). In certain embodiments, the target miRNA is selected from the group consisting of miR-1, miR-24, miR-32, miR-103, miR-107, miR122, miR-124, miR-125, miR-127, miR-128, miR-130, miR-132, miR-134, miR-135, miR-138, miR-143, miR-148, miR-150, miR-151, miR-152, miR-153, miR-181, miR-189, miR-192, miR-194, miR-195, miR-199, miR-203, miR-204, miR-206, miR-208, miR-212, miR-215, miR-216, miR-221, miR-222, miR-375, miR-378, miR-30b, miR-30c, miR-122a, miR-133a, miR-200a, miR-142-3p, miR-143-5p, let-7, and a viral microRNA.

In certain embodiments, the target miRNA is miR-1. In certain embodiments, the target miRNA is miR-122. In certain embodiments, the target miRNA is miR-192. In certain embodiments, the target miRNA is miR-203. In certain embodiments, the target miRNA is miR-208.

In certain embodiments, the target miRNA is a miRNA expressed in muscle tissue. In certain embodiments, the muscle tissue is skeletal muscle and/or cardiac muscle. In certain embodiments, the target miRNA is a miRNA expressed in neuronal tissue.

In certain embodiments, the target miRNA is a miRNA expressed in a specific tissue within a multicellular organism. In certain embodiments, the multicellular organism is a mammal. In certain embodiments, the multicellular organism is a plant.

In certain embodiments, the miRNA exhibits a tissue specific expression pattern.

In certain embodiments, the target mRNA is expressed in skeletal muscle and the target miRNA is miR-1. In certain embodiments, the target mRNA is expressed in cardiac muscle and the target miRNA is miR-208. In certain embodiments, the target mRNA is expressed in liver and the target miRNA is miR-122. In certain embodiments, the target mRNA is expressed in kidney and the target miRNA is miR-192. In certain embodiments, the target mRNA is expressed in skin and the target miRNA is miR-203.

The RNA-modulating agents can have a variety of architectures. If the RNA-modulate agent comprises more than one miRNA binding sequence, then the miRNA binding sequences can be linked together (i.e., in series or branching) on one end of the RNA-modulating agent (linked to either the 3′ or 5′ end of the mRNA binding sequence). Alternatively the miRNA binding sequences can be on either side of (i.e., flanking) the mRNA binding sequence in the RNA-modulating agent. In certain embodiments, the miRNA binding sequences are linked together in series in the RNA-modulating agent. In certain embodiments, the miRNA binding sequences are linked to the same end of the mRNA molecule in parallel branches in the RNA-modulating agent. In certain embodiments, the mRNA binding sequence is flanked by miRNA binding sequences in the RNA-modulating agent. In certain embodiments, the miRNA binding sequences are linked together in series, and linked to the 5′ end of the mRNA binding sequence. In certain embodiments, the miRNA binding sequences are linked together in series, and linked to the 3′ end of the mRNA binding sequence. In certain embodiments, the RNA-modulating agent is circular (e.g., a circular oligonucleotide).

RNA-modulating agents may comprise one more modified nucleotides. A number of nucleotide and nucleoside modifications have been shown to make an oligonucleotide more resistant to nuclease digestion, thereby prolonging in vivo half-life. Specific examples of modified oligonucleotides include those comprising backbones comprising, for example, phosphorothioates, phosphotriesters, methyl phosphonates, short chain alkyl or cycloalkyl intersugar linkages or short chain heteroatomic or heterocyclic intersugar linkages. In certain embodiments, the RNA-modulating agent comprises one or more phosphorothioate internucleotide linkages (i.e., backbones) and those with heteroatom backbones, particularly CH₂—NH—O—CH₂, CH, ˜N(CH₃)˜O˜CH₂ (known as a methylene(methylimino) or MMI backbone), CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497), each of which is herein incorporated by reference in its entirety. Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,405,939; 5,453,496; 5,455,233; 5,466,677; 5,476,925; 5,519,126; 5,541,306; 5,550,111; 5,563,253; 5,571,799; 5,587,361; and 5,625,050, each of which is herein incorporated by reference in its entirety. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol, 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991, each of which is herein incorporated by reference in its entirety. Cyclohexenyl nucleic acid oligonucleotide mimetics are described in Wang et al., J. Am. Chem. Soc., 2000, 122, 8595-8602, the contents of which is incorporated herein in its entirety.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts; see U.S. Pat. Nos. 5,034,506; 5,166,315; 5,185,444; 5,214,134; 5,216,141; 5,264,562; 5,264,564; 5,405,938; 5,434,257; 5,466,677; 5,470,967; 5,541,307; 5,561,225; 5,596,086; 5,602,240; 5,610,289; 5,602,240; 5,610,289; 5,618,704; 5,623,070; 5,663,312; 5,633,360; 5,677,437; and each of which is herein incorporated by reference in its entirety.

In certain embodiments, RNA-modulating agents comprise one or more substituted sugar moieties, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃ where n is from 1 to about 10; Ci to CIO lower alkyl, alkoxyalkoxy, substituted lower alkyl, alkaryl or aralkyl; CI; Br; CN; CF3; OCF3; O—, S—, or N-alkyl; O—, S—, or N-alkenyl; SOCH3; SO2 CH₃; ONO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacokinetic/pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al., Helv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. “Locked” nucleic acids (LNA) may also be used in which the 2′ hydroxyl of the ribose sugar is connected, e.g., by a methylene or ethylene bridge, to the 4′ carbon of the same ribose sugar (e.g., a C2′-O,C4′-ethylene-bridged nucleotide). The sugar group can also contain one or more carbons that possess the opposite stereochemical configuration than that of the corresponding carbon in ribose. Thus, a modified RNA can include nucleotides containing e.g., arabinose, as the sugar. Oligonucleotides may also have sugar mimetics such as cyclobutyls in place of the pentofuranosyl group.

In certain embodiments, RNA-modulating agents comprise one or more base modifications and/or substitutions. As used herein, “unmodified” or “natural” bases include adenine (A), guanine (G), thymine (T), cytosine (C) and uracil (U). Modified bases include, without limitation, bases found only infrequently or transiently in natural nucleic acids, e.g., N⁶-methyladenosine (m⁶A), pseudouridine, hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic bases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions can also be included. These have been shown to increase nucleic acid duplex stability by 0.6-1.2 OC. (Sanghvi, Y. S., in Crooke, S. T. and Lebleu, B., eds., Antisense Research and Applications, CRC Press, Boca Raton, 1993, pp. 276-278). Further suitable modified bases are described in U.S. Pat. No. 3,687,808, as well as U.S. Pat. Nos. 4,845,205; 5,130,302; 5,175,273; 5,367,066; 5,432,272; 5,457,187; 5,459,255; 5,484,908; 5,525,711; 5,552,540; 5,587,469; 5,596,091; 5,614,617; 5,750,692, and each of which is herein incorporated by reference.

It is not necessary for all positions in a given oligonucleotide to be uniformly modified, and in fact more than one of the aforementioned modifications may be incorporated in a single oligonucleotide or even at within a single nucleoside within an oligonucleotide.

In certain embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; and 5,719,262, each of which is herein incorporated by reference. Further teaching of PNA compounds can be found in Nielsen et al., Science, 1991, 254, 1497-1500.

### Conjugated Functional Moieties

In other embodiments, the RNA-modulating agents may be modified with one or more functional moieties. A functional moiety is a molecule that confers one or more additional activities to the RNA-modulating agent. In certain embodiments, the functional moieties enhance cellular uptake by target cells (e.g., neuronal cells). Thus, the disclosure includes RNA-modulating agents which are conjugated or unconjugated (e.g., at its 5′ and/or 3′ terminus) to another moiety (e.g. a non-nucleic acid moiety such as a peptide), an organic compound (e.g., a dye), or the like. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

In a certain embodiment, the functional moiety is a hydrophobic moiety. In a certain embodiment, the hydrophobic moiety is selected from the group consisting of fatty acids, steroids, secosteroids, lipids, gangliosides and nucleoside analogs, endocannabinoids, and vitamins. In a certain embodiment, the steroid selected from the group consisting of cholesterol and Lithocholic acid (LCA). In a certain embodiment, the fatty acid selected from the group consisting of Eicosapentaenoic acid (EPA), Docosahexaenoic acid (DHA) and Docosanoic acid (DCA). In a certain embodiment, the vitamin selected from the group consisting of choline, vitamin A, vitamin E, and derivatives or metabolites thereof. In a certain embodiment, the vitamin is selected from the group consisting of retinoic acid and alpha-tocopheryl succinate.

In a certain embodiment, an RNA-modulating agent of disclosure is conjugated to a lipophilic moiety. In one embodiment, the lipophilic moiety is a ligand that includes a cationic group. In certain embodiments, the lipophilic moiety is selected from the group consisting of cholesterol, vitamin E, vitamin K, vitamin A, folic acid, a cationic dye (e.g., Cy3). In an exemplary embodiment, the lipophilic moiety is cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

In certain embodiments, the functional moieties may comprise one or more ligands tethered to an RNA-modulating agent to improve stability, hybridization thermodynamics with a target nucleic acid, targeting to a particular tissue or cell-type, or cell permeability, e.g., by an endocytosis-dependent or -independent mechanism. Ligands and associated modifications can also increase sequence specificity and consequently decrease off-site targeting.

Ligands can include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine (GalNAc) or derivatives thereof, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycodies, artificial endonucleases (e.g. EDTA), lipophilic molecules, e.g, cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C₁₀, C₁₁, C₁₂, C₁₃, C₁₄, C₁₅, C₁₆, C₁₇, Cia, C₁₉, or C₂₀ fatty acids) and ethers thereof, e.g., C₁₀, C₁₁, C₁₂, C₁₃, C₁₄, C₁₅, C₁₆, C₁₇, C₁₈, C₁₉, or C₂₀ alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu³⁺ complexes of tetraazamacrocycles), dinitrophenyl, HRP or AP. In certain embodiments, the ligand is GalNAc or a derivative thereof.

In certain embodiments, the functional moiety is linked to the RNA-modulating agent by a linker.

In certain embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments, the linker is a cleavable linker.

In certain embodiments, the linker comprises a dTdT dinucleotide.

The various functional moieties of the disclosure and means to conjugate them to RNA-modulating agents are described in further detail in WO2017/030973A1 and WO2018/031933A2, incorporated herein by reference.

### III. Methods of Use

In one aspect, the disclosure provides a method of treating a subject having a disease or disorder characterized by or caused by: (a) the overexpression or overactivity of a normal cellular protein; (b) the underexpression or underactivity of a normal cellular protein; (c) the activity of a mutant protein; or (d) the activity of a viral RNA or protein, the method comprising administering to the subject an effective amount of an RNA-modulating agent discloses herein, wherein the RNA-modulating agent binds to the mRNA encoding the protein and modulates expression of a protein.

The RNA-modulating agents are useful for modulating the expression of mRNA in a variety of tissues, including, but not limited to, muscle tissue (e.g., skeletal and/or cardiac), liver, skin, and kidneys.

In another aspect, the disclosure provides a method of modulating the expression of a neuromuscular target mRNA in a subject, the method comprising administering to the subject an effective amount of an RNA-modulating agent, wherein the RNA-modulating agent comprises an mRNA binding sequence that is complementary to a portion of the neuromuscular target mRNA, linked to one or more miRNA binding sequences, wherein the one or more miRNA binding sequences are complementary to at least positions 2 to 8 of a miRNA, and wherein the RNA-modulating agent binds to the neuromuscular target mRNA, thereby modulating the expression of the neuromuscular target mRNA.

In another aspect, the disclosure provides a method of modulating the expression of a target mRNA in skeletal muscle in a subject, the method comprising administering to the subject an effective amount of an RNA-modulating agent, wherein the RNA-modulating agent comprises an mRNA binding sequence that is complementary to a portion of the target mRNA, linked to one or more miR-1 binding sequences, wherein the one or more miRNA binding sequences are complementary to at least positions 2 to 8 of miR-1, and wherein the RNA-modulating agent binds to the target mRNA, thereby modulating the expression of the target mRNA in the skeletal muscle in the subject.

In another aspect, the disclosure provides a method of modulating the expression of a target mRNA in cardiac muscle in a subject, the method comprising administering to the subject an effective amount of an RNA-modulating agent, wherein the RNA-modulating agent comprises an mRNA binding sequence that is complementary to a portion of the target mRNA, linked to one or more miR-208 binding sequences, wherein the one or more miRNA binding sequences are complementary to at least positions 2 to 8 of miR-208, and wherein the RNA-modulating agent binds to the target mRNA, thereby modulating the expression of the target mRNA in the cardiac muscle in the subject.

In another aspect, the disclosure provides a method of modulating the expression of a target mRNA in kidney in a subject, the method comprising administering to the subject an effective amount of an RNA-modulating agent, wherein the RNA-modulating agent comprises an mRNA binding sequence that is complementary to a portion of the target mRNA, linked to one or more miR-192 binding sequences, wherein the one or more miRNA binding sequences are complementary to at least positions 2 to 8 of miR-192, and wherein the RNA-modulating agent binds to the target mRNA, thereby modulating the expression of the target mRNA in the kidney in the subject.

In another aspect, the disclosure provides a method of modulating the expression of a target mRNA in skin in a subject, the method comprising administering to the subject an effective amount of an RNA-modulating agent, wherein the RNA-modulating agent comprises an mRNA binding sequence that is complementary to a portion of the target mRNA, linked to one or more miR-203 binding sequences, wherein the one or more miRNA binding sequences are complementary to at least positions 2 to 8 of miR-203, and wherein the RNA-modulating agent binds to the target mRNA, thereby modulating the expression of the target mRNA in the skin in the subject.

In another aspect, the disclosure provides a method of modulating the expression of a target mRNA in liver in a subject, the method comprising administering to the subject an effective amount of an RNA-modulating agent, wherein the RNA-modulating agent comprises an mRNA binding sequence that is complementary to a portion of the target mRNA, linked to one or more miR-122 binding sequences, wherein the one or more miRNA binding sequences are complementary to at least positions 2 to 8 of miR-122, and wherein the RNA-modulating agent binds to the target mRNA, thereby modulating the expression of the target mRNA in the liver in the subject.

The miRNA binding sequences of the RNA-modulating agents in the methods described herein comprise at least one modified nucleotide at nucleotide positions that are complementary to at one or more of positions 2, 5, and 8 of the miRNA. In other embodiments, the miRNA binding sequence comprises a modified nucleotide at nucleotide positions that are complementary to positions 2, 5, and 8 of the miRNA.

In certain embodiments, the modified nucleotide is selected from a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide.

In certain embodiments, the modified nucleotide comprises an LNA or a PNA.

In certain embodiments, the RNA-modulating agent is capable of discriminatory binding of the target miRNA relative to a non-target miRNA.

In certain embodiments, the non-target miRNA sequence differs from the target miRNA sequence by at least one nucleotide.

In another aspect, the disclosure provides a method of treating or preventing sarcopenia and/or cachexia in a subject, the method comprising administering to the subject an effective amount of an RNA-modulating agent,
wherein the RNA-modulating agent comprises an mRNA binding sequence that is complementary to a portion of an activin A mRNA, linked to a miRNA binding sequence,
wherein the miRNA binding sequence is complementary to at least positions 2 to 8 of a target miRNA, and
wherein the RNA-modulating agent binds to the activin A mRNA, thereby treating or preventing sarcopenia and/or cachexia in the subject.

In certain embodiments, the target miRNA is miR-1. In certain embodiments, the miRNA binding sequence comprises, from 5′ to 3′, ACAUUCCA. In certain embodiments, the mRNA binding sequence comprises, from 5′ to 3′, CUGUCUUCUCUGGAC(SEQ ID NO: 1). In certain embodiments, the RNA-modulating agent comprises, from 5′ to 3′, ACAUUCCACUGUCUUCUCUGGAC(SEQ ID NO:2). In certain embodiments, the RNA-modulating agent comprises, from 5′ to 3′,

(SEQ ID NO: 3)
(lA)#(mC)#(mA)#(lT)(mU)(mC)(lC)(mA)(mC)
(mU)(lG)(mU)(mC)(lT)(mU)(mC)(lT)(mC)(mU)
(lG)#(mG)#(mA)#(lC),

wherein “l” corresponds to an LNA modification, “m” corresponds to a 2′-O-methyl modification, and “#” corresponds to a phosphorothioate internucleotide linkage.

### EXAMPLES

The present invention is further illustrated by the following examples which should not be construed as further limiting. The contents of Sequence Listing, figures and all references, patents and published patent applications cited throughout this application are expressly incorporated herein by reference.

Furthermore, in accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

### Example 1: Discriminatory RNA-Modulating Agents

The instant disclosure describes RNA-modulating agents with a mRNA binding sequence and a miRNA binding sequence. The miRNA binding sequence comprises at least one modified nucleotide at nucleotide positions that are complementary to one or more of positions 2, 5, and 8 of a miRNA. The use of chemical modifications, LNA and PNA in particular, facilitate discriminatory binding of the target miRNA relative to a non-target miRNA. The non-target miRNA sequence may differ from the target miRNA sequence by as little as one nucleotide, and the RNA-modulating agents will preferentially bind the target miRNA.

To demonstrate this effect, several RNA-modulating agents were designed, employing LNA modifications at position 2, 5, and 8.

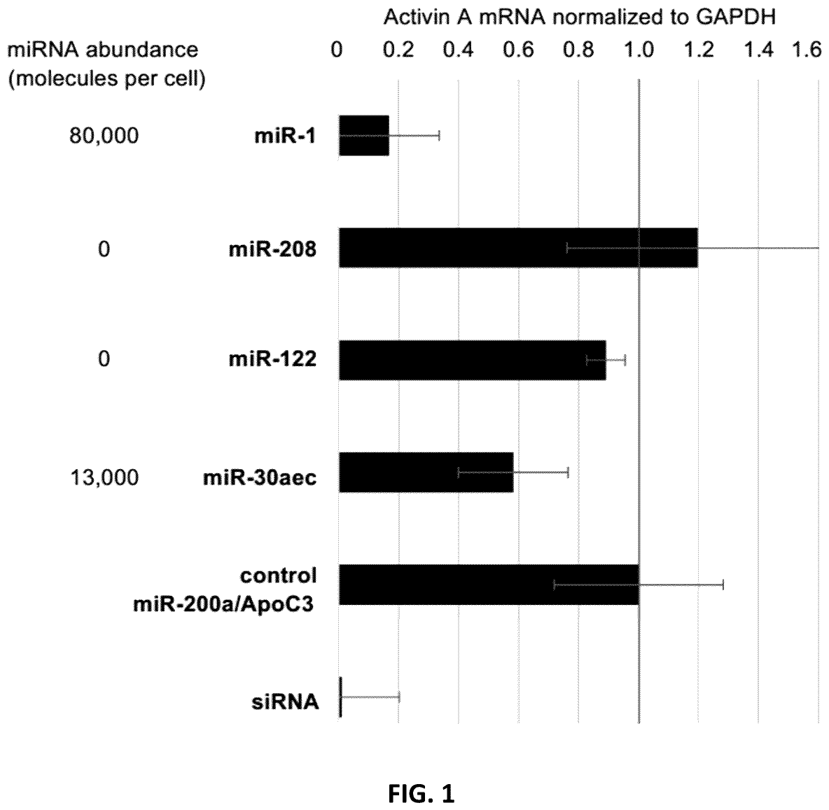

As shown in FIG. 1, activin A mRNA was targeted with an RNA-modulating agent in C2C12 mouse myotubes. C2C12 mouse myotubes were transfected with 20 nM of the RNA-modulating agent targeting activin A mRNA for silencing using different miRNAs. Abundance of Activin A mRNA was measured by qRT-PCR 72 h post transfection and normalized to GAPDH and control tether. Data was normalized to transfection efficiency based on percent of cells expressing yellow fluorescent protein. Data is the mean±standard deviation from three independent replicates.

The RNA-modulating agent using a miR-1 binding sequence achieved high level silencing of activin A compared to other miR binding sequences. MiR-1 has approximately 80,000 copies in the C2C12 cells, whereas other tested microRNAs (miR-122 and miR-208) have none.

The following RNA-modulating agents were used in FIG. 1:

MiR-1 targeting:
(SEQ ID NO: 3)
(lA)#(mC)#(mA)#(lT)(mU)(mC)(lC)(mA)(mC)(mU)
(lG)(mU)(mC)(lT)(mU)(mC)(lT)(mC)(mU)(lG)#(mG)
#(mA)#(lC)
MiR-208 targeting:
(SEQ ID NO: 4)
(lT)(mU)(lT)(mU)(lT)(mC)(mG)(mA)(lC)(mG)(lT)
(mC)(lT)(mU)(lA)(mU)(mC)(mU)(lG)(mU)(mC)(lT)
(mU)(mC)(lT)(mC)(mU)(lG)(mG)(mA)(lC)
MiR-122 targeting:
(SEQ ID NO: 5)
(lC)(mC)(lA)(mU)(lT)(mA)(mG)(mA)(lA)(mC)(lA)
(mC)(lT)(mC)(lC)(mA)(mC)(mU)(lG)(mU)(mC)(lT)
(mU)(mC)(lT)(mC)(mU)(lG)(mG)(mA)(lC)
MiR-1 seed plus 11-16/ActA:
(SEQ ID NO: 6)
5′(lA)(mC)(lT)(mU)(lC)(mU)(mA)(mA)(lA)(mC)(lA)
(mU)(lT)(mC)(lC)(mA)(mC)(mU)(lG)(mU)(mC)(lT)
(mU)(mC)(lT)(mC)(mU)(lG)(mG)(mA)(lC)-3′
MiR-30aec seed plus 12-16/ActA:
(SEQ ID NO: 7)
(lG)(mU)(lC)(mG)(lA)(mU)(mU)(mU)(lT)(mG)(lT)
(mU)(lT)(mA)(lC)(mA)(mC)(mU)(lG)(mU)(mC)(lT)
(mU)(mC)(lT)(mC)(mU)(lG)(mG)(mA)(lC)
MiR-200a seed plus 12-16. LNA t2, t14/ApoC3:
(SEQ ID NO: 8)
(mU)(mU)(lA)(mC)(mC)(mU)(mU)(mU)(mC)(mA)(mG)
(mU)(mG)(mU)(lT)(mA)(lA)(mG)(lC)(mA)(lG)(mC)
(lT)(mU)(lC)(mU)(lT)(mG)(mU)(lC)(mC)
Activin A guide strand:
(SEQ ID NO: 9)
/5Phos/rUrC rCrUrC rArCrU rArUrA rArUrC rCrAr
G rCrArA Rc
Activin A passenger strand:
(SEQ ID NO: 10)
rUrGrC rUrGrG rArUrU rArUrA rGrUrG rArGrG
rCrUrU
“l” corresponds to an LNA modification,
“m” corresponds to a 2′-O-methyl
modification, “#” corresponds to a
phosphorothioate internucleotide linkage.

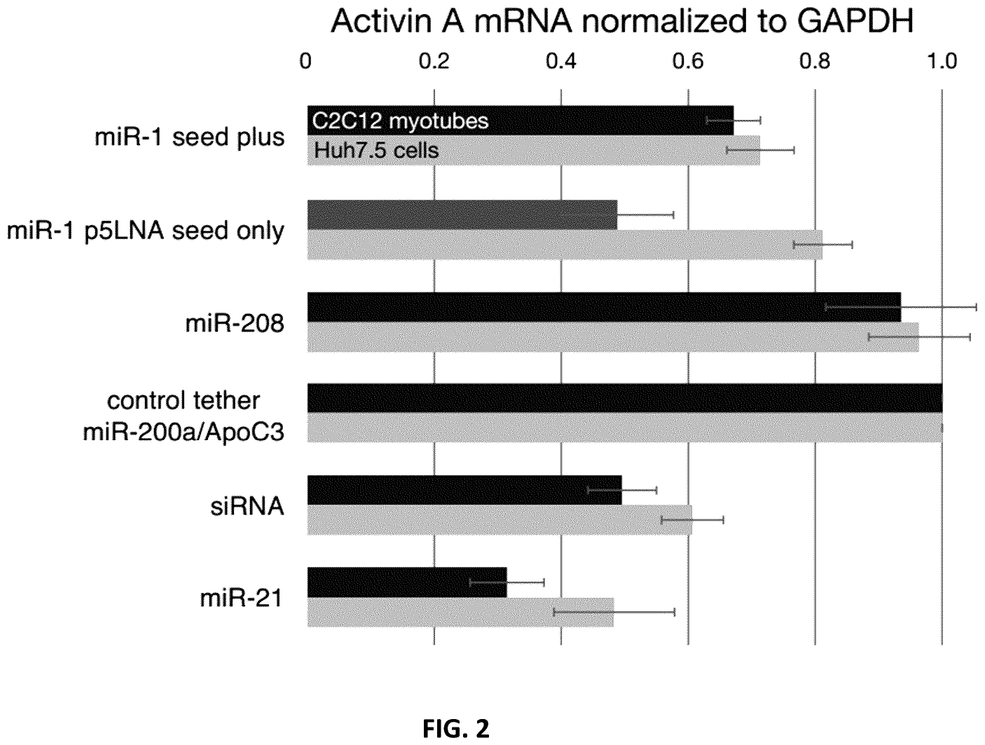

A similar experiment was performed, as shown in FIG. 2. Activin A mRNA levels were measured and normalized to GAPDH in C2C12 mouse myotubes and Huh7.5 human hepatocytes (bottom bar) incubated with an RNA-modulating agent. Cells were transfected with 20 nM of each RNA-modulating agent. Abundance of Activin A mRNA was measured by qRT-PCR 72 h post transfection and normalized to GAPDH and control tether. Data is the mean±standard error from three independent replicate. The data shows that tethers without an LNA at the nucleotide that pairs with position 5 of the seed sequence of miR-1 silenced the target in both cell types, suggesting that the tether binds miR-122 in hepatocytes that do not express miR-1. Tethers with LNA at the nucleotide that pairs with position 5 of the seed sequence of miR-1 silenced the target in myotubes that express miR-1, but not in hepatocytes.

miR-1 and miR-208 targeting RNA-modulating agents were next used in vivo to demonstrate tissue specific targeting between skeletal muscle, cardiac muscle, and liver tissue. Female C57bl/6 mice, aged 23 months, received 4 interscapular subcutaneous doses of 10 mg/kg of the miR-1 targeting RNA-modulating agent (tether A) or the miR-208 targeting RNA-modulating agent (tether B). 28 days post-injection the mass of each quadriceps muscle was measured and normalized to body weight.

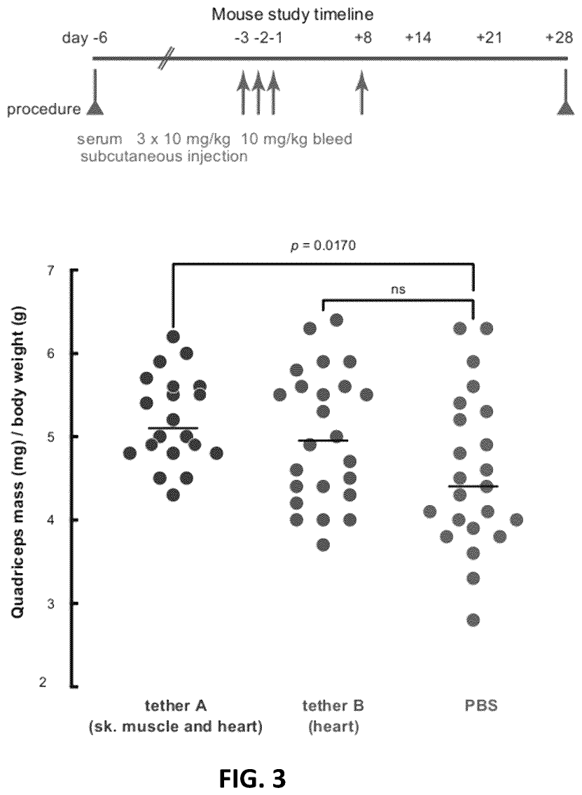

As shown in FIG. 3, the group (n=20) that received tether A had a significantly increased sarcopenic index for quadriceps mass (mg)/body weight (g) of 5.2 (p=0.017, one-way ANOVA, Dunnett's multiple comparisons test) compared to 4.6 in the control group (n=23). The tether B group (n=24) had a sarcopenic index of 5.0 (p=0.106). Black bar indicates median.

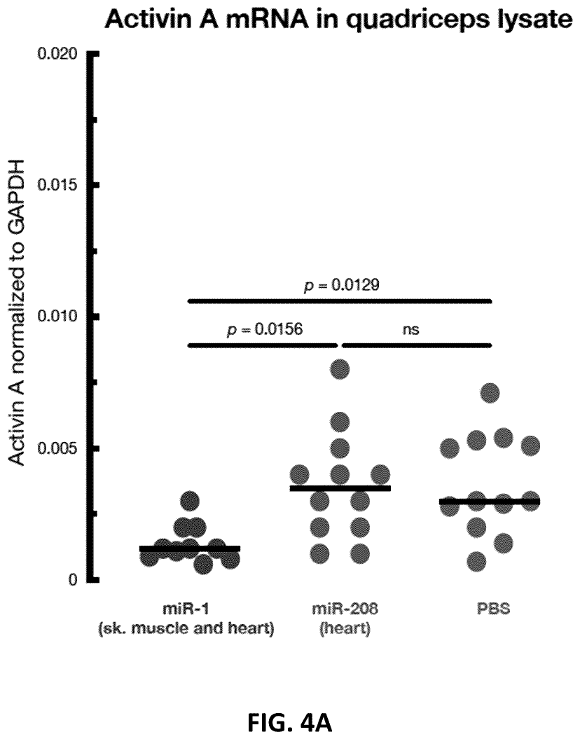

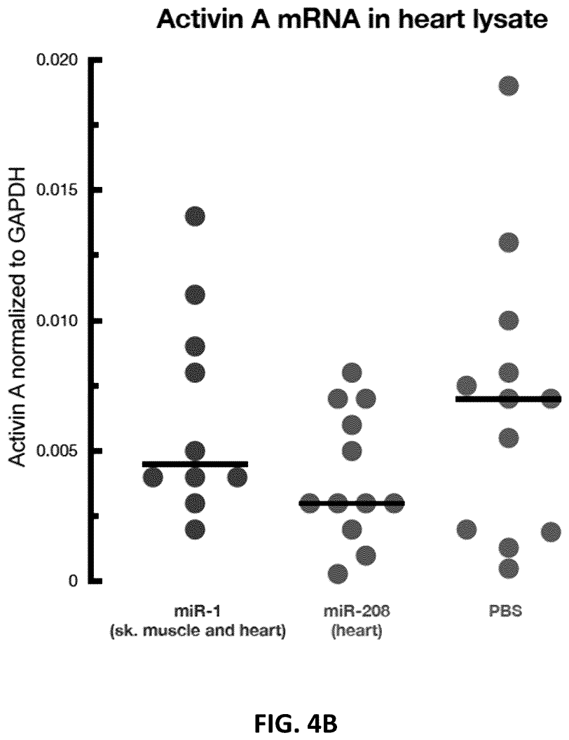

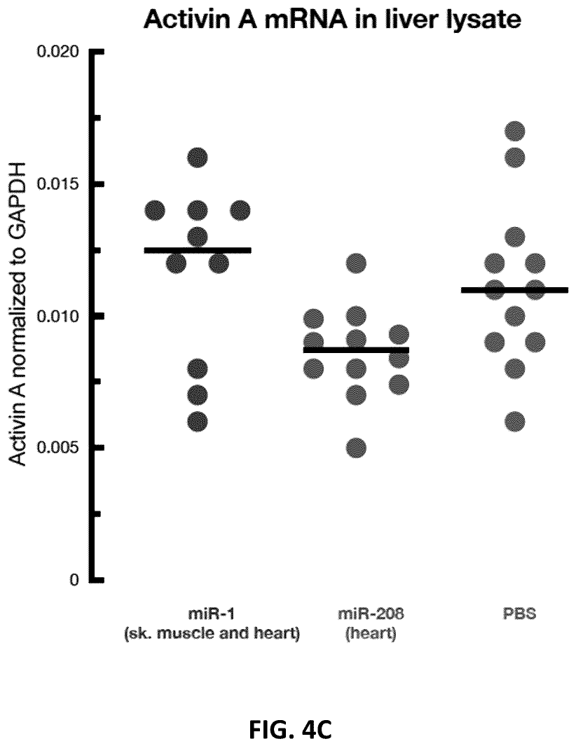

As shown in FIGS. 4A-C, miR-1 tether significantly decreased activin A mRNA in quadriceps muscle lysate. The miR-208 tether did not reduce activin A in quadriceps lysate because it should only silence activin A in heart tissue. Activin A mRNA was not significantly decreased in heart lysate after 28 days. A 3-month study may detect a change in activin A levels in heart tissue. Due to liver exposure of the DCA-conjugated tethers, the level of activin A mRNA in liver lysate was assess but significant Activin A silencing in liver was not detected. One-way ANOVA, Tukey's multiple comparisons adjusted p value.

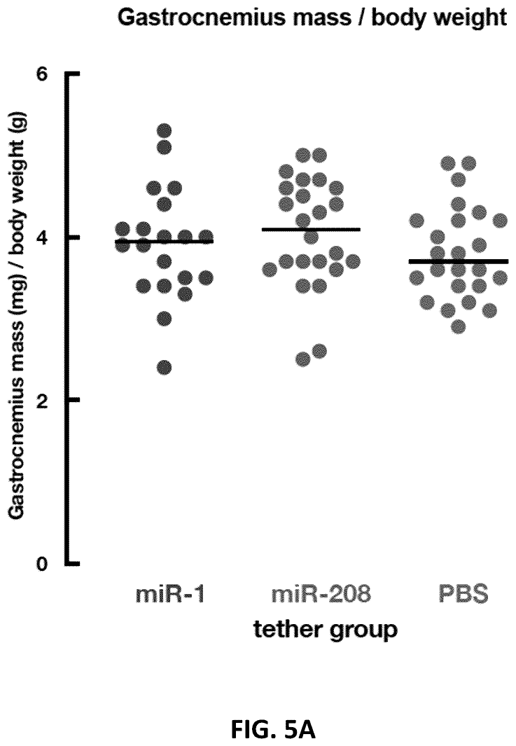

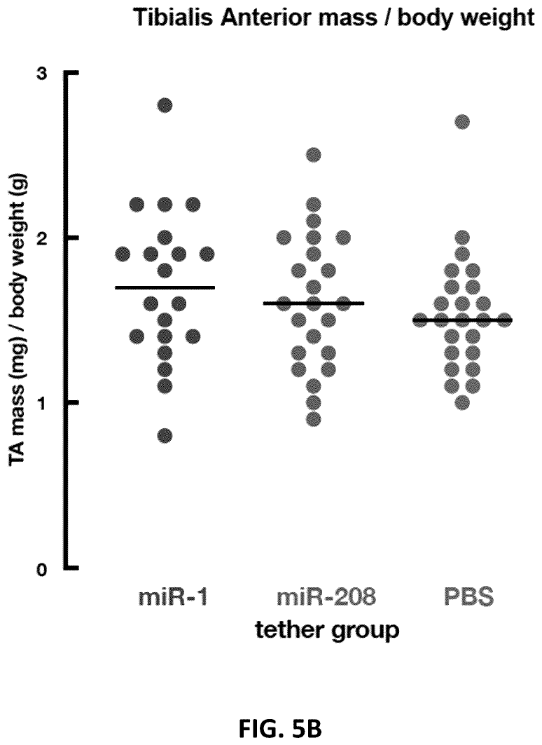

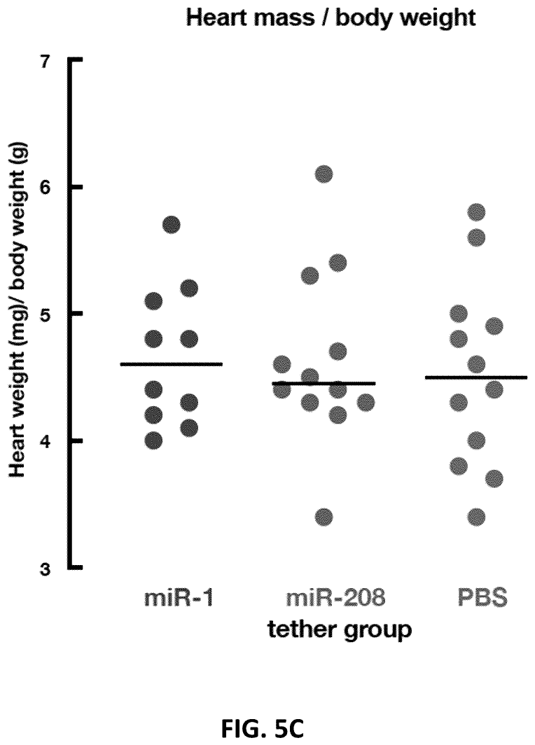

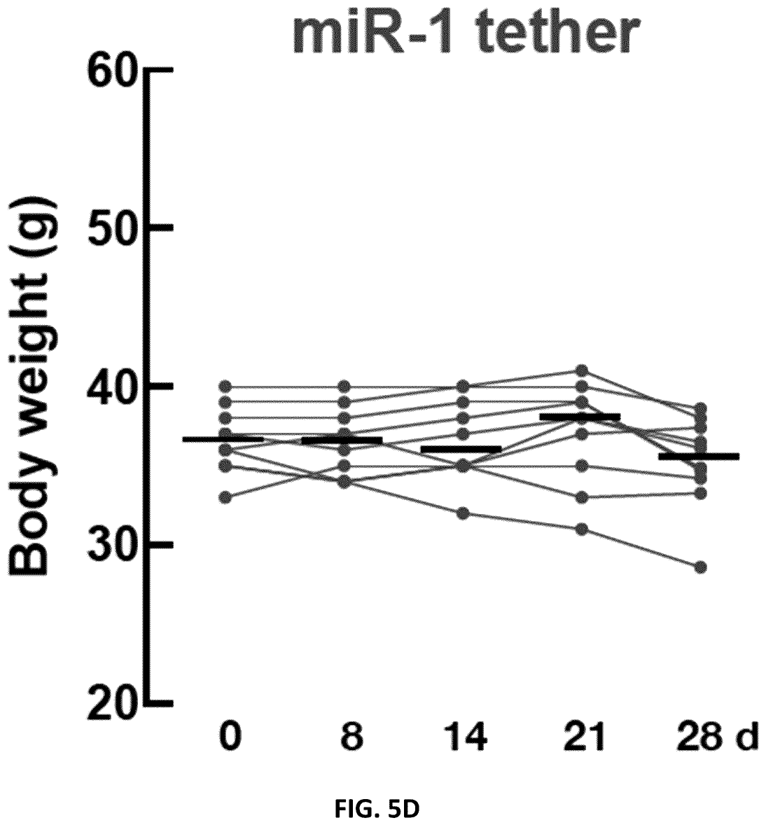

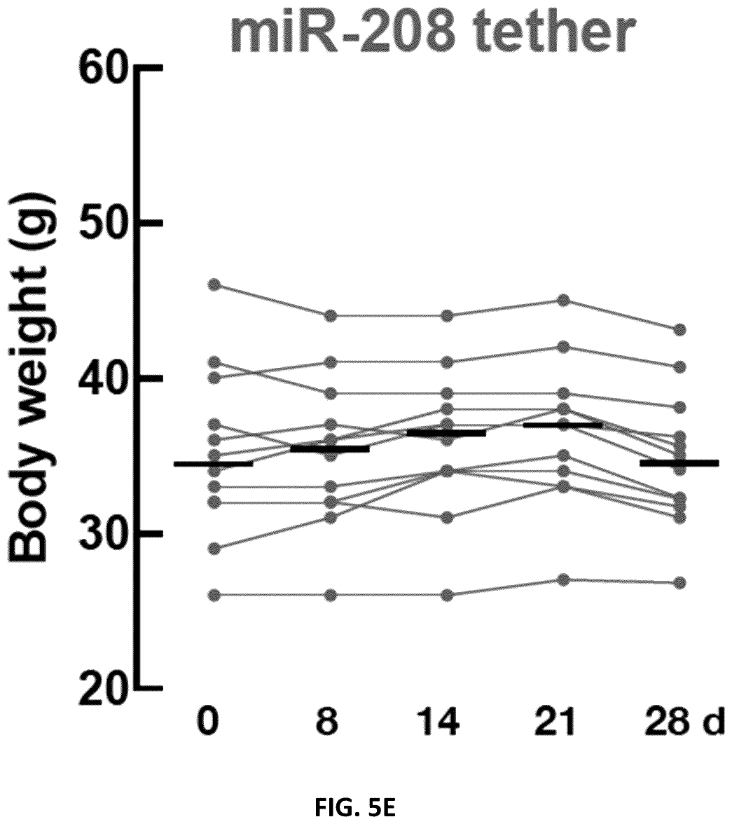

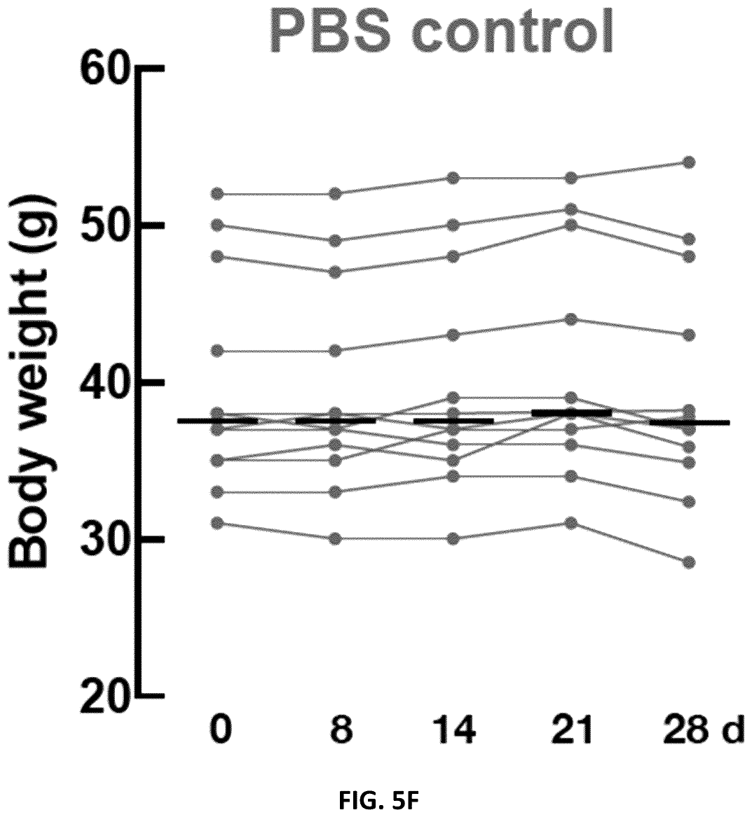

As shown in FIGS. 5A-F, mice gastrocnemius mass relative level (FIG. 5A), tibialis anterior mass relative level (FIG. 5B), heart mass relative level (FIG. 5C), and body weight over time (FIGS. 5D-F) after miR-1 and miR-208 injections were assessed.

The following RNA-modulating agents were used in FIGS. 2-5:

MiR-1 targeting (tether A):
(SEQ ID NO: 24)
(lA)#(mC)#(mA)#(lT)(mU)(mC)(lC)(mA)(mC)(mU)
(lG)(mU)(mC)(lT)(mU)(mC)(lT)(mC)(mU)(lG)#(mG)#
(mA)#(lC)(dTdT)-DCA
MiR-208 targeting (tether B):
(SEQ ID NO: 25)
(lC)#(mG)#(lT)#(mC)(lT)(mU)(lA)(mU)(mC)(mU)
(lG)(mU)(mC)(lT)(mU)(mC)(lT)(mC)(mU)(lG)#(mG)#
(mA)#(lC)(dTdT)-DCA
MiR-1 seed plus 11-16:
(SEQ ID NO: 6)
(lA)(mC)(lT)(mU)(lC)(mU)(mA)(mA)(lA)(mC)(lA)
(mU)(lT)(mC)(lC)(mA)(mC)(mU)(lG)(mU)(mC)(lT)
(mU)(mC)(lT)(mC)(mU)(lG)(mG)(mA)(lC)
MiR-1 p5 LNA seed only:
(SEQ ID NO: 11)
(lA)(mC)(mA)(lT)(mU)(mC)(lC)(mA)(mC)(mU)(lG)
(mU)(mC)(lT)(mU)(mC)(lT)(mC)(mU)(lG)(mG)
(mA)(lC)
MiR-208 seed plus 12-16/ActA:
(SEQ ID NO: 4)
(lT)(mU)(lT)(mU)(lT)(mC)(mG)(mA)(lC)(mG)(lT)
(mC)(lT)(mU)(lA)(mU)(mC)(mU)(lG)(mU)(mC)(lT)
(mU)(mC)(lT)(mC)(mU)(lG)(mG)(mA)(lC)
MiR-200a seed plus 12-16, LNA t2, t14/ApoC3:
(SEQ ID NO: 8)
(mU)(mU)(lA)(mC)(mC)(mU)(mU)(mU)(mC)(mA)(mG)
(mU)(mG)(mU)(lT)(mA)(lA)(mG)(lC)(mA)(lG)(mC)
(lT)(mU)(lC)(mU)(lT)(mG)(mU)(lC)(mC)
Activin A guide strand:
(SEQ ID NO: 9)
/5Phos/rUrC rCrUrC rArCrU rArUrA rArUrC
rCrArG rCrArA rC
Activin A passenger strand:
(SEQ ID NO: 10)
rUrGrC rUrGrG rArUrU rArUrA rGrUrG
rArGrG rCrUrU
miR-21 seed only/ActA:
(SEQ ID NO: 12)
(lA)(mU)(lA)(mA)(lG)(mC)(lT)(mA)(mC)(mU)
(lG)(mU)(mC)(lT)(mU)(mC)(lT)(mC)(mU)(lG)
(mG)(mA)(lC)
“l” corresponds to an LNA modification,
“m” corresponds to a 2′-O-methyl
modification, “#” corresponds to a
phosphorothioate internucleotide linkage,
“dTdT” corresponds to a dTdT dinucleotide
linker, “DCA” corresponds to a docosanoic
acid (DCA) conjugate, and “r” corresponds
to RNA.

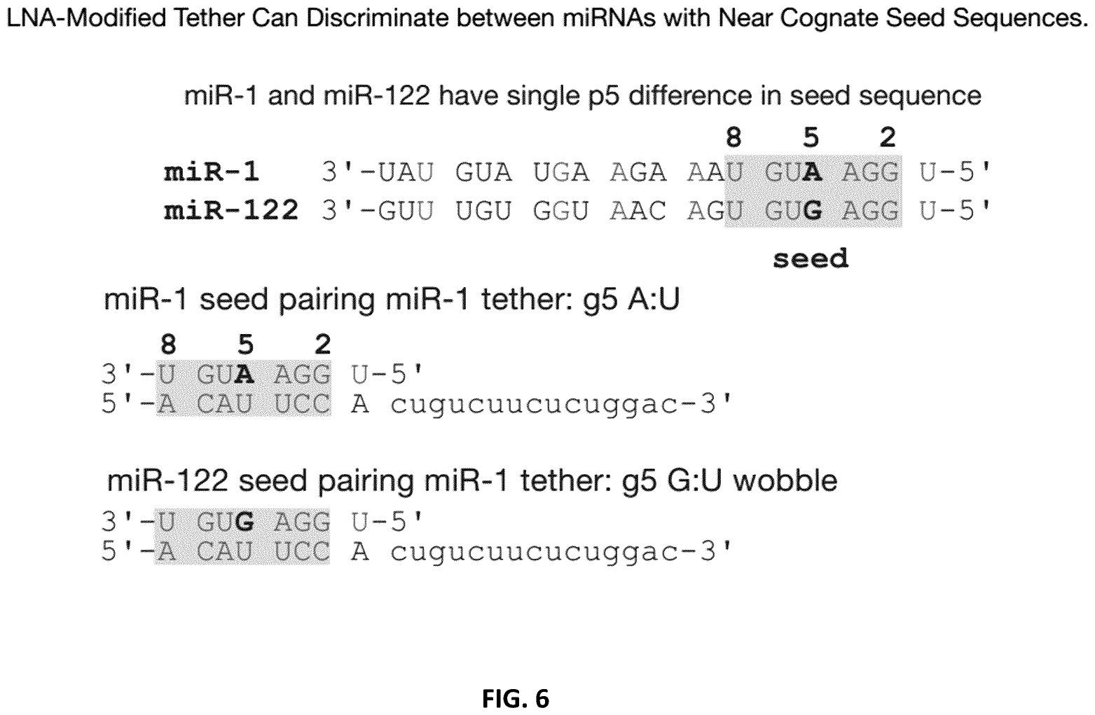

FIG. 6 depicts locked nucleic acid (LNA) modified RNA-modulating agent discrimination between miRNAs with near cognate seed sequences.

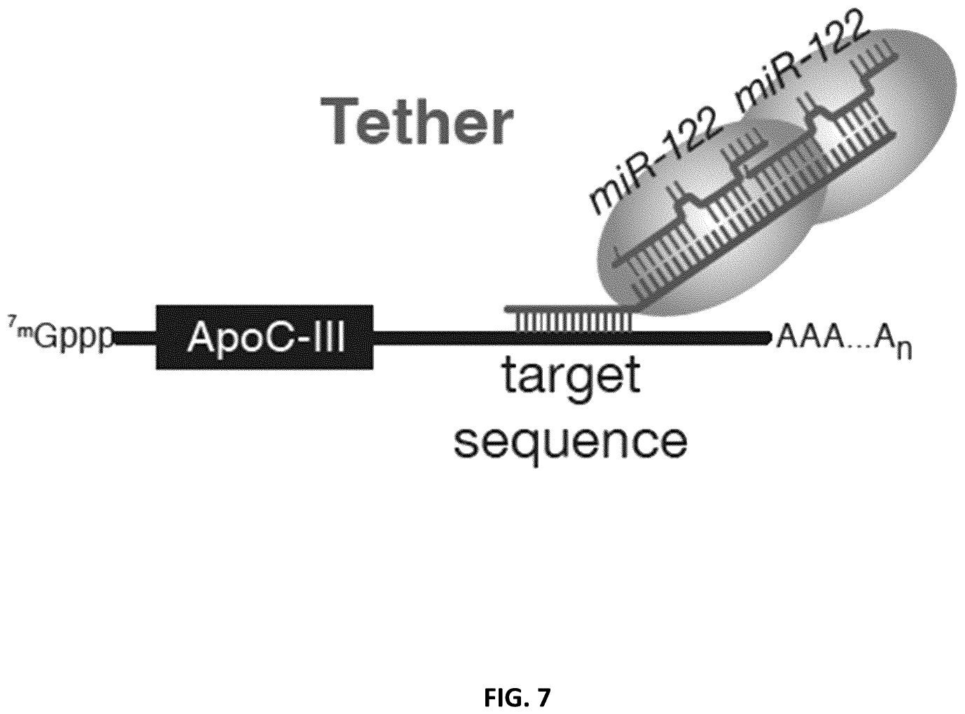

Another similar experiment was performed, as shown in FIGS. 7-10. A tether was used to bring miR-1221 RISC to a target such as a single 15 nt site in 3′ UTR of ApoC3 as illustrated in FIG. 7.

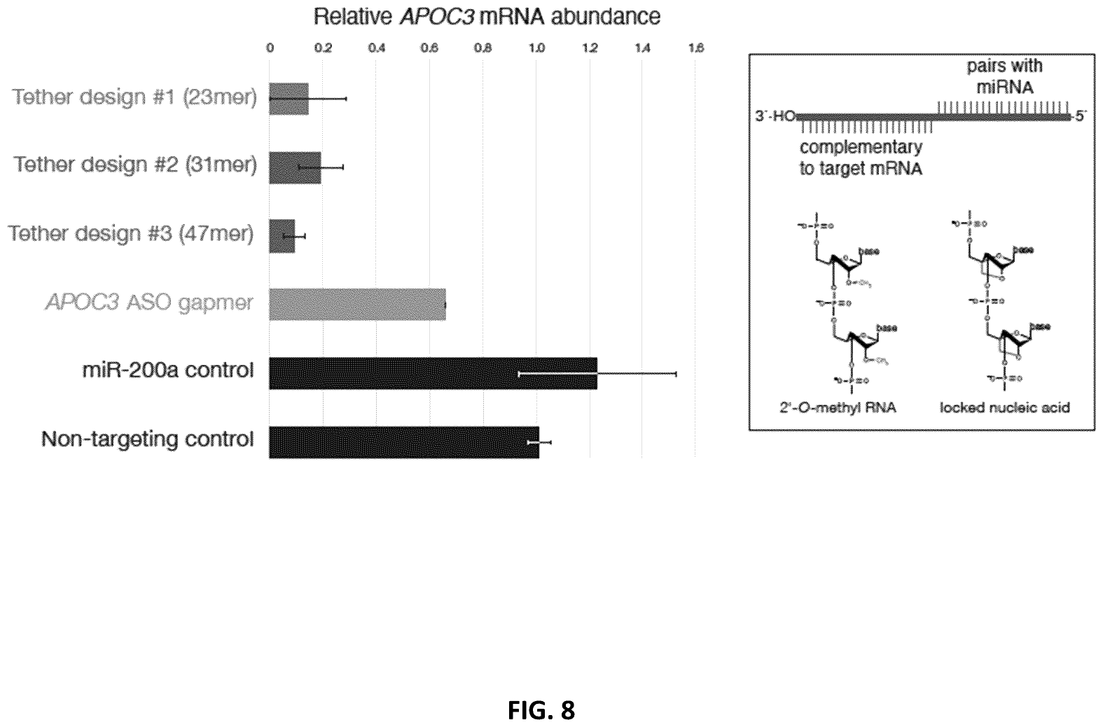

As shown in FIG. 8, three oligonucleotide tethers and a tether complementary to miR-200a were assessed. The three oligonucleotide tethers were designed to recruit miR-122 to ApoC3 mRNA and were found to reduce ApoC3 mRNA abundance in hepatocyte-derived Huh7.5 cells. In contrast, the tether complementary to miR-200a is a miRNA not expressed in Huh7.5 cells and was found to do not decrease ApoC3 mRNA abundance in hepatocyte-derived Huh7.5 cells. Huh7.5 cells were co-transfected with nM tether oligonucleotides and 1 μg pEYFP plasmid (TransIT-X2, MirusBio). RNA was harvested (RNAeasy Plus, Qiagen) 72 h later. Transfection efficiency was determined in parallel by measuring the percent of cells expressing YFP (MACSQuant® VYB, Miltenyi Biotech). APOC3 and GAPDH mRNA levels were measured by RT-qPCR.

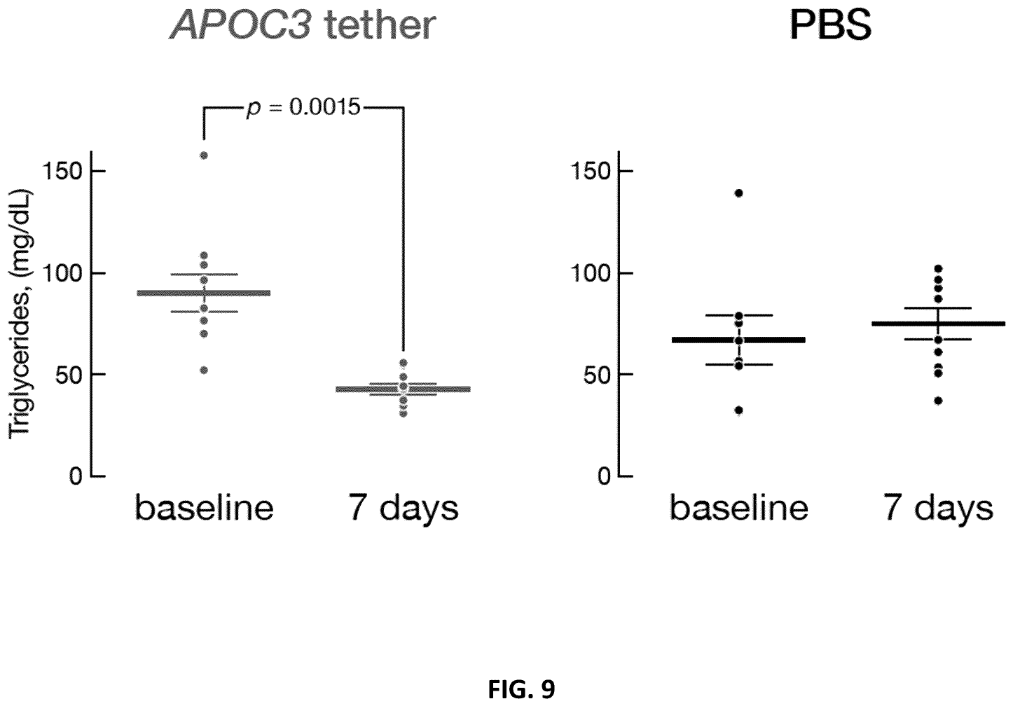

As shown in FIG. 9, an oligonucleotide tether designed to bind miR-122 and APOC3 mRNA was assessed. The oligonucleotide was found to significantly reduce serum triglycerides in vivo in mice. The 3′ end of the tether (tether design #3) was conjugated to GalNAc to facilitate delivery to hepatocytes. Mice received 3.3 mg per kg tether by interscapular sub-cutaneous injection on days 1, 2, and 3. Serum was collected 10 days before the first injection (baseline) and 7 days after the last injection. Statistical significance was measured with a paired, two-tailed Student's t-test.

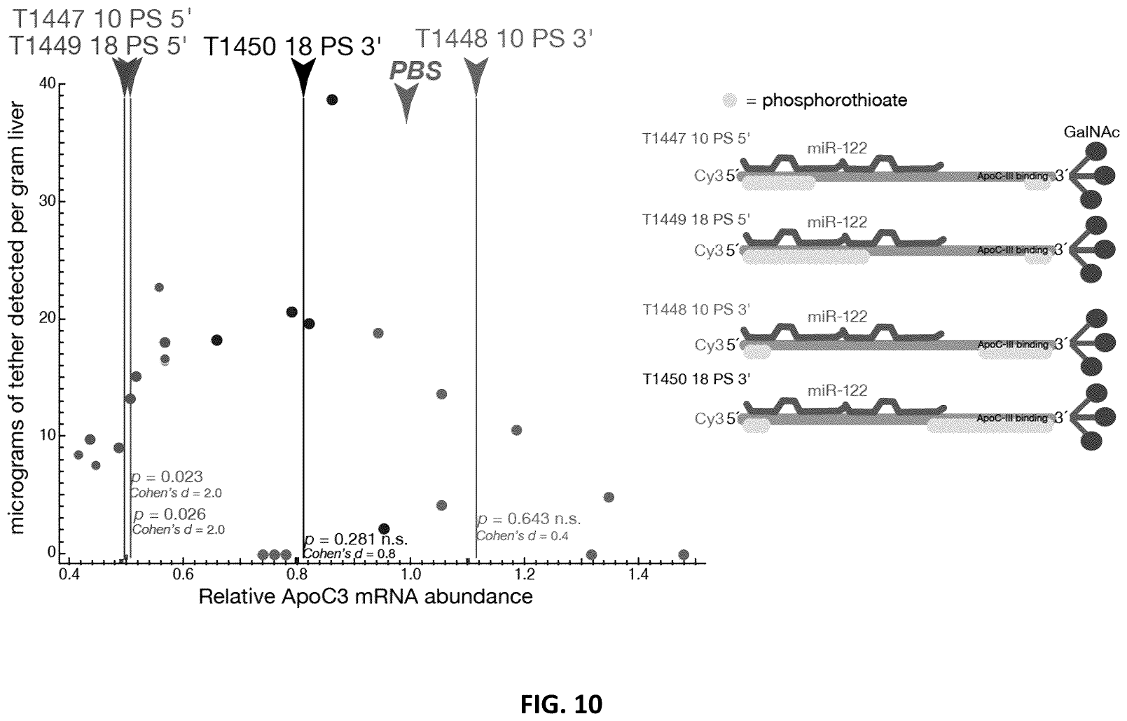

As shown in FIG. 10, the activity of ApoC3 tethers to reduce ApoC3 mRNA in mouse liver was assessed. Tethers with 10 or 18 phosphorothioate linkages in the 5′ end of the tether was found to reduce mRNA levels by 50% (blue and red arrowheads). Tethers with 10 or 18 phosphorothioate linkages in the 3′ end of the tether, next to the GalNAc, was found to do not reduce mRNA levels (n=5 C57Bl/6 mice per group). The 3′ end of the tether (tether design #3) was conjugated to GalNAc to facilitate delivery to hepatocytes. Mice received a single 10 mg per kg tether by interscapular sub-cutaneous injection on day 1. The amount of tethers and ApoC3 mRNA in liver tissue was measured by Quantigene Assay after 48 hours. Circles indicate amount of tether detected in liver lysate (micrograms of tether per gram of liver, color of circle corresponds to the tether received, red, 1447; blue, 1449; green, 1448; black, 1450; grey, PBS. Statistical significance was measured with an unpaired, two-tailed Student's t-test.

The following RNA-modulating agents were used in FIGS. 8-10:

Tether design #1 (23mer)-
(SEQ ID NO: 13)
T LNA 1xmiR-122 seed/ApoC3 L15B:
(lA)(mC)(lA)(mC)(lT)(mC)(lC)(mA)(lA)(mG)(lC)
(mA)(lG)(mC)(lT)(mU)(lC)(mU)(lT)(mG)(mU)#(lC)
#(mC)
Tether design #2 (31mer)-
T LNA 1xmiR-122 12-16/ApoC3 L15B:
(SEQ ID NO: 14)
(lC)#(mC)#(lA)(mU)(lT)(mA)(mG)(mA)(lA)(mC)
(lA)(mC)(lT)(mC)(lC)(mA)(lA)(mG)(lC)(mA)(lG)
(mC)(lT)(mU)(lC)(mU)(lT)(mG)(mU)#(lC)#(mC)
Tether design #3 (47mer)-T.APOC3
2′ O-methyl 2x miR-12seed plus 12-16/
2′ O-methy 15 ApoC3:
(SEQ ID NO: 15)
(mC)(mC)(mA)(mU)(mU)(mA)(mG)(mA)(mA)(mC)(mA)
(mC)(mU)(mC)(mC)(mA)(mC)(mC)(mA)(mU)(mU)(mA)
(mG)(mA)(mA)(mC)(mA)(mC)(mU)(mC)(mC)(mA)(mA)
(mG)(mC)(mU)(mU)(mC)(mU)(mU)(mG)(mU)(mC)(mC)
(mA)(mG)(mC)
APOC3 ASO gapmer:
(SEQ ID NO: 16)
(mA)(mG)(mC)(mU)(mU)(mC)(T)(T)(G)(T)(C)(C)
(A)(G)(C)(mU)(mU)(mU)(mA)(mU)
miR-200a control-LNA miR-200a/LNA ApoC3:
(SEQ ID NO: 8)
(mU)(mU)(lA)(mC)(mC)(mU)(mU)(mU)(mC)(mA)(mG)
(mU)(mG)(mU)(lT)(mA)(lA)(mG)(lC)(mA)(lG)(mC)
(lT)(mU)(lC)(mU)(lT)(mG)(mU)(lC)(mC)
Non-targeting control-Control LNA 1xT9A
12-16 miR122/15 CXCR4:
(SEQ ID NO: 17)
(lC)#(mC)#(lA)(mU)(lT)(mA)(mG)(mA)(lA)(mC)
(lA)(mC)(lT)(mC)(lC)(mA)(mC)(mC)(mG)(mG)(mU)
(mG)(mU)(mU)(mA)(mG)(mC)(mU)(mU)#(mU)#(mG)
Fluorescently labeled GalNAc tethers to
ApoC3-1449-567-18 PS 5′ Cy3:
(SEQ ID NO: 18)
Cy3-(mC)#(mC)#
(mA)#(mU)#(mU)#(mA)#(mG)#(mA)#(mA)#
(mC)#(mA)#(mC)#(mU)#(mC)#(mC)#(mA)#(mC)#(mC)#
(mA)(mU)(mU)(mA)(mG)(mA)(mA)(mC)(mA)(mC)(mU)
(mC)(mC)(mA)(mA)(mG)(mC)(mU)(mU)(mC)(mU)(mU)
(mG)(mU)(mC)(mC)#(mA)#(mG)#(mC)-GalNAc
Fluorescently labeled GalNAc tethers to
ApoC3-1450-567-18 PS 3′ Cy3:
(SEQ ID NO: 19)
Cy3-(mC)#(mC)#
(mA)#(mU)(mU)(mA)(mG)(mA)(mA)(mC)(m
A)(mC)(mU)(mC)(mC)(mA)(mC)(mC)(mA)(mU)(mU)(mA
)(mG)(mA)(mA)(mC)(mA)(mC)(mU)#(mC)#(mC)#(mA)#
(mA)#(mG)#(mC)#(mU)#(mU)#(mC)#(mU)#(mU)#(mG)#
(mU)#(mC)#(mC)#(mA)#(mG)#(mC)-GalNAc
Fluorescently labeled GalNAc tethers to
ApoC3-1447-567-10 PS 5′ Cy3:
(SEQ ID NO: 20)
Cy3-(mC)#(mC)#
(mA)#(mU)#(mU)#(mA)#(mG)#(mA)#(mA)#
(mC)#(mA)(mC)(mU)(mC)(mC)(mA)(mC)(mC)(mA)(mU)
(mU)(mA)(mG)(mA)(mA)(mC)(mA)(mC)(mU)(mC)(mC)
(mA)(mA)(mG)(mC)(mU)(mU)(mC)(mU)(mU)(mG)(mU)
(mC)(mC)#(mA)#(mG)#(mC)-GalNAc
Fluorescently labeled GalNAc tethers
to ApoC3-1448-567-10 PS 3′ Cy3:
(SEQ ID NO: 21)
Cy3-(mC)#(mC)#(mA)#(mU)(mU)(mA)(mG)(mA)(mA)(mC)
(mA)(mC)(mU)(mC)(mC)(mA)(mC)(mC)(mA)(mU)(mU)(mA)
(mG)(mA)(mA)(mC)(mA)(mC)(mU)(mC)(mC)(mA)(mA)
(mG)(mC)(mU)(mU)#(mC)#(mU)#(mU)#(mG)#(mU)#(mC)
#(mC)#(mA)#(mG)#(mC)-GalNAc
“l” corresponds to an LNA modification,
“m” corresponds to a 2′-O-methyl
modification, “#” corresponds to a
phosphorothioate internucleotide linkage.

1. An RNA-modulating agent comprising an mRNA binding sequence that is complementary to a portion of a target mRNA sequence, linked to one or more miRNA binding sequences,
wherein the one or more miRNA binding sequences is complementary to at least positions 2 to 8 of a miRNA from the 5′ end of the miRNA, and
wherein the miRNA binding sequence comprises at least one modified nucleotide at a nucleotide position that is complementary to any one or more of positions 2, 5, and 8 of the miRNA from the 5′ end of the miRNA.

2. The RNA-modulating agent of claim 1, wherein the miRNA binding sequence comprises a modified nucleotide at nucleotide positions that are complementary to positions 2, 5, and 8 of the miRNA.

3. The RNA-modulating agent of claim 1, wherein:
the modified nucleotide is selected from a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide;
the modified nucleotide comprises an LNA or a PNA;
the RNA-modulating agent reduces target mRNA abundance by at least 50% in a tissue;
the RNA-modulating agent is capable of discriminatory binding of the target miRNA relative to a non-target miRNA;
the non-target miRNA sequence differs from the target miRNA sequence by at least one nucleotide;
the RNA modulating agent comprises one miRNA binding sequence;
the RNA modulating agent comprises two or more miRNA binding sequences;
the target mRNA is a neuromuscular mRNA target;
the target miRNA is a miRNA expressed in muscle tissue;
the muscle tissue is skeletal muscle and/or cardiac muscle;
the target miRNA is a miRNA expressed in neuronal tissue;
the target miRNA is a miRNA expressed in a specific tissue within a multicellular organism;
multicellular organism is a mammal;
multicellular organism is a plant; and/or
the miRNA exhibits a tissue specific expression pattern.

4-17. (canceled)

18. The RNA-modulating agent of claim 1, wherein:
the one or more miRNA binding sequences are not complementary to positions 10 and 11 of the miRNA;
the one or more miRNA binding sequences are complementary to positions 2 to 8, and 12 to 15, 12 to 16, or 12 to 17 of the miRNA, but not complementary to positions 10 and 11 of the miRNA;
the one or more miRNA binding sequences are not complementary to position 1 of the miRNA;
the one or more miRNA binding sequences are complementary to positions 2 to 8, and 12 to 15, 12 to 16, or 12 to 17 of the miRNA, but not complementary to positions 1, 10 and 11 of the miRNA;
the miRNA binding sequences are complementary to only positions 2 to 8 of the miRNA; or
the one or more miRNA binding sequences have an adenosine, at a position in the miRNA binding sequence corresponding to position 9 of the miRNA.

19-23. (canceled)

24. The RNA-modulating agent of claim 1, wherein:
the one or more miRNA binding sequences are about 8 to about 25 nucleotides in length;
the one or more miRNA binding sequences are 8 nucleotides in length; and/or
the mRNA binding sequence is about 15 nucleotides in length.

25-26. (canceled)

27. The RNA-modulating agent of claim 1, wherein:
the target miRNA is selected from the group consisting of miR-1, miR-24, miR-32, miR-103, miR-107, miR122, miR-124, miR-125, miR-127, miR-128, miR-130, miR-132, miR-134, miR-135, miR-138, miR-143, miR-148, miR-150, miR-151, miR-152, miR-153, miR-181, miR-189, miR-192, miR-194, miR-195, miR-199, miR-203, miR-204, miR-206, miR-208, miR-212, miR-215, miR-216, miR-221, miR-222, miR-375, miR-378, miR-30b, miR-30c, miR-122a, miR-133a, miR-200a, miR-142-3p, miR-143-5p, let-7, and a viral microRNA;
the target miRNA is miR-1;
the target miRNA is miR-122;
the target miRNA is miR-192;
the target miRNA is miR-203; or
the target miRNA is miR-208.

28-32. (canceled)

33. The RNA-modulating agent of any one of claim 1, wherein:
a functional moiety is linked to the 5′ end and/or 3′ end of the RNA-modulating agent;
a functional moiety is linked to the 3′ end of the RNA-modulating agent;
the functional moiety comprises a hydrophobic moiety;
the hydrophobic moiety is selected from the group consisting of fatty acids, steroids, secosteroids, lipids, gangliosides, nucleoside analogs, endocannabinoids, vitamins, and a mixture thereof;
the steroid selected from the group consisting of cholesterol and Lithocholic acid (LCA);
the fatty acid selected from the group consisting of Eicosapentaenoic acid (EPA), Docosahexaenoic acid (DHA) and Docosanoic acid (DCA);
the vitamin is selected from the group consisting of choline, vitamin A, vitamin E, and derivatives or metabolites thereof;
the vitamin is selected from the group consisting of retinoic acid and alpha-tocopheryl succinate;
the functional moiety is linked to the RNA-modulating agent by a linker;
the linker comprises an ethylene glycol chain, an alkyl chain, a peptide, an RNA, a DNA, a phosphodiester, a phosphorothioate, a phosphoramidate, an amide, a carbamate, or a combination thereof;
the linker is a cleavable linker; and/or
the linker comprises a dTdT dinucleotide.

34-44. (canceled)

45. The RNA-modulating agent of claim 1, wherein:
the target mRNA is expressed in skeletal muscle and the target miR-1;
the target mRNA is activin A;
the mRNA binding sequence comprises CUGUCUUCUCUGGAC (SEQ ID NO: 1); and/or
the one or more miRNA binding sequences comprises ACAUUCCA.

46-48. (canceled)

49. The RNA-modulating agent of claim 1, wherein:
the target mRNA is expressed in cardiac muscle and the target miRNA is miR-208;
the target mRNA is MARK4, VASH1, or VASH2;
the target mRNA is expressed in liver and the target miRNA is miR-122;
the target mRNA is ApoC3;
the target mRNA is expressed in kidney and the target miRNA is miR-192;
the target mRNA is Smad3;
the target mRNA is expressed in skin and the target miRNA is miR-203; and/or
the target mRNA is elastase, cathepsin K, or a matrix metallo-proteinase (MMP) mRNA (e.g., MMP-1, MMP-8, MMP-13, MMP-14, MMP-16, and MMP-18).

50-56. (canceled)

57. The RNA-modulating agent of claim 1, comprising a chemical modification pattern of
(lN)#(mN)#(mN)#(lN)(mN)(mN)(lN)(mN)(mN)(mN)(lN)(mN)(mN)(lN)(mN)(mN)(lN)(mN)(m N)(lN)#(mN)#(mN)#(lN),
wherein “l” corresponds to an LNA modification, “m” corresponds to a 2′-O-methyl modification, “#” corresponds to a phosphorothioate internucleotide linkage, and “N” corresponds to any nucleotide (A, T, U, G, or C).

58. A method of treating a subject having a disease or disorder characterized by or caused by:
(a) the overexpression or overactivity of a normal cellular protein;
(b) the underexpression or underactivity of a normal cellular protein;
(c) the activity of a mutant protein; or
(d) the activity of a viral RNA or protein,
the method comprising administering to the subject an effective amount of an RNA-modulating agent of claim 1, wherein the RNA-modulating agent binds to the mRNA encoding the protein and modulates expression of a protein.

59. A method of modulating the expression of a target mRNA in a subject, the method comprising administering to the subject an effective amount of an RNA-modulating agent, wherein:
A:
the target mRNA is a neuromuscular target mRNA,
the RNA-modulating agent comprises an mRNA binding sequence that is complementary to a portion of the neuromuscular target mRNA, linked to one or more miRNA binding sequences,
the one or more miRNA binding sequences are complementary to at least positions 2 to 8 of a miRNA, and
the RNA-modulating agent binds to the neuromuscular target mRNA, thereby modulating the expression of the neuromuscular target mRNA;
B:
the target mRNA is in skeletal muscle in the subject,
the RNA-modulating agent comprises an mRNA binding sequence that is complementary to a portion of the target mRNA, linked to one or more miR-1 binding sequences,
the one or more miRNA binding sequences are complementary to at least positions 2 to 8 of miR-1, and
the RNA-modulating agent binds to the target mRNA, thereby modulating the expression of the target mRNA in the skeletal muscle in the subject;
C:
the target mRNA is in cardiac muscle in the subject,
the RNA-modulating agent comprises an mRNA binding sequence that is complementary to a portion of the target mRNA, linked to one or more miR-208 binding sequences,
the one or more miRNA binding sequences are complementary to at least positions 2 to 8 of miR-208, and
the RNA-modulating agent binds to the target mRNA, thereby modulating the expression of the target mRNA in the cardiac muscle in the subject;
D:
the target mRNA is in kidney in the subject,
the RNA-modulating agent comprises an mRNA binding sequence that is complementary to a portion of the target mRNA, linked to one or more miR-192 binding sequences,
the one or more miRNA binding sequences are complementary to at least positions 2 to 8 of miR-192, and
the RNA-modulating agent binds to the target mRNA, thereby modulating the expression of the target mRNA in the kidney in the subject;
E:
the target mRNA is in skin in the subject,
the RNA-modulating agent comprises an mRNA binding sequence that is complementary to a portion of the target mRNA, linked to one or more miR-203 binding sequences,
the one or more miRNA binding sequences are complementary to at least positions 2 to 8 of miR-203, and
the RNA-modulating agent binds to the target mRNA, thereby modulating the expression of the target mRNA in the skin in the subject; or
F:
the target mRNA is in liver in the subject,
the RNA-modulating agent comprises an mRNA binding sequence that is complementary to a portion of the target mRNA, linked to one or more miR-122 binding sequences,
the one or more miRNA binding sequences are complementary to at least positions 2 to 8 of miR-122, and
the RNA-modulating agent binds to the target mRNA, thereby modulating the expression of the target mRNA in the liver in the subject.

60-64. (canceled)

65. The method of claim 59, wherein:
the miRNA binding sequence comprises at least one modified nucleotide at nucleotide positions that are complementary to at one or more of positions 2, 5, and 8 of the miRNA; or
the miRNA binding sequence comprises a modified nucleotide at nucleotide positions that are complementary to positions 2, 5, and 8 of the miRNA.

66. (canceled)

67. The method of claim 59, wherein:
the modified nucleotide is selected from a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide;
the modified nucleotide comprises an LNA or a PNA;
the RNA-modulating agent is capable of discriminatory binding of the target miRNA relative to a non-target miRNA,
the non-target miRNA sequence differs from the target miRNA sequence by at least one nucleotide;
the method comprises one miRNA binding sequence; and/or
the method comprises two or more miRNA binding sequences.

68-72. (canceled)

73. A method of treating or preventing sarcopenia and/or cachexia in a subject, the method comprising administering to the subject an effective amount of an RNA-modulating agent,
wherein the RNA-modulating agent comprises an mRNA binding sequence that is complementary to a portion of an activin A mRNA, linked to a miRNA binding sequence,
wherein the miRNA binding sequence is complementary to at least positions 2 to 8 of a target miRNA, and
wherein the RNA-modulating agent binds to the activin A mRNA, thereby treating or preventing sarcopenia and/or cachexia in the subject.

74. The method of claim 73, wherein the miRNA binding sequence comprises at least one modified nucleotide at one or more of positions 2, 5, and 8 of the miRNA.

75. The method of claim 73, wherein:
the target miRNA is miR-1;
the miRNA binding sequence comprises, from 5′ to 3′, ACAUUCCA;
the mRNA binding sequence comprises, from 5′ to 3′, CUGUCUUCUCUGGAC (SEQ ID NO: 1);
the RNA-modulating agent comprises, from 5′ to 3′, ACAUUCCACUGUCUUCUCUGGAC (SEQ ID NO: 2); and/or
the RNA-modulating agent comprises, from 5′ to 3′,
(lA)#(mC)#(mA)#(lT)(mU)(mC)(lC)(mA)(mC)(mU)(lG)(mU)(mC)(lT)(mU)(mC)(lT)(mC)(mU) (lG)#(mG)#(mA)#(lC) (SEQ ID NO: 3),
wherein “l” corresponds to an LNA modification, “m” corresponds to a 2′-O-methyl modification, and “#” corresponds to a phosphorothioate internucleotide linkage.

76-79. (canceled)

80. An RNA-modulating agent comprising an mRNA binding sequence that is complementary to a portion of an Activin A mRNA sequence, linked to one or more miRNA binding sequences.

81. The RNA-modulating agent of claim 80, wherein the one or more miRNA binding sequences is complementary to at least positions 2 to 8 of a miRNA from the 5′ end of the miRNA.

82. The RNA-modulating agent of claim 80, wherein:
the miRNA binding sequence comprises at least one modified nucleotide at a nucleotide position that is complementary to any one or more of positions 2, 5, and 8 of the miRNA from the 5′ end. of the miRNA;
wherein the miRNA binding sequence comprises at least one modified nucleotide at one or more of positions 2, 5, and 8 of the miRNA;
the target miRNA is miR-1;
the miRNA binding sequence comprises, from 5′ to 3′, ACAUUCCA;
the mRNA binding sequence comprises, from 5′ to 3′, CUGUCUUCUCUGGAC;
the RNA-modulating agent comprises, from 5′ to 3′, ACAUUCCACUGUCUUCUCUGGAC (SEQ ID NO: 2);
the RNA-modulating agent comprises, from 5′ to 3′,
(lA)#(mC)#(mA)#(lT)(mU)(mC)(lC)(mA)(mC)(mU)(lG)(mU)(mC)(lT)(mU)(mC)(lT)(mC)(mU) (lG)#(mG)#(mA)#(lC) (SEQ ID NO: 3),
wherein “l” corresponds to an LNA modification, “m” corresponds to a 2′-O-methyl modification, and “#” corresponds to a phosphorothioate internucleotide linkage.

83-88. (canceled)
